SEQUENCE LISTING (2 sequences)

SEQ ID NO: 1 - DNA, 4 bp, Artificial Sequence
  synthetic polynucleotide
ttgc

SEQ ID NO: 2 - DNA, 4 bp, Artificial Sequence
  synthetic polynucleotide
atgc

(19) United States
(12) Patent Application Publication
(10) Pub. No.: US 2005/0244821 A1
(43) Pub. Date: Nov. 3, 2005

(54) Method of identification and quantification of biological molecules and apparatus therefore

(76) Inventors: Ory Zik, Tel Aviv (IL); Elisha Moses, Rehovot (IL); Nava Levit-Binnun, Tel Aviv (IL); Ariel B. Lindner, Tel Aviv (IL)

Correspondence Address: LADAS & PARRY, 26 WEST 61ST STREET, NEW YORK, NY, 10023, US

(21) Appl. No.: 10/344,650

(22) PCT Filed: Aug. 16, 2001
(86) PCT No.: PCT/IL01/00764

§ 371 Date: Jul. 27, 2004

Related U.S. Application Data
(60) Provisional application No. 60/225,747, filed on Aug. 17, 2000.

(51) Int. Cl. C12Q 1/68; G01N 33/53

(52) U.S. Cl. 435/6; 435/7.1

(57) ABSTRACT

A method of detecting binding between first member or members of a binding pair and corresponding second member or members of the binding pair is disclosed. The method comprises interacting a solid support onto which the first member or members of the binding pair being immobilized and arrayed with the corresponding second member or members of the binding pair, the corresponding second member or members of the binding pair being directly or indirectly tagged with a heavy atom; and determining a spatial distribution of the heavy atom over a surface of the solid support, thereby detecting the binding between the first member or members of the binding pair and the corresponding second member or members of the binding pair.

### FIELD OF THE INVENTION

The present invention relates to a method and apparatus for identifying and quantifying molecules present in a sample, in particular by means of irradiating appropriately tagged molecules with a particle beam, such as an electron beam, obtaining an image of the tags and carrying out image analysis. The present invention find uses and provides major improvements in the fields of genomics, proteomics, functional proteomics, glycomics and cellomics.

### BACKGROUND OF THE INVENTION

In the past, genes, proteins, carbohydrates and cells where mainly studied at isolation, greatly limiting the ability to elucidate a realistic picture of the complex array of biochemical processes taking place in living cells. Genomics, proteomics, glycomics and cellomics techniques, which evolved in this sequence during the last decade, aim at analyzing biochemical processes, as complex as these may be, in a more integrated fashion, aiming at looking at all or substantially all of the biochemical changes, large scale changes, as well as minute changes, that take place in living cells under various conditions.

Presently, genomics, proteomics, glycomics and cellomics rely on several technologies that are insufficiently quantitative, insufficiently sensitive and are characterized by a relatively low signal-to-noise (S/N) ratio and as such fail to provide a complete insight of cell function. These include the nowadays routine technology of nucleic acid microarrays (e.g., DNA microarrays, also referred to in the art as DNA chips) for analyzing nucleic acid molecules, such as DNA and RNA; the emerging technology of protein (e.g., antigen or antibody) microarrays (also known as protein chips); the recently revived and improved technology of two-dimensional polyacrylamide gel electrophoresis (2D-PAGE), both latter techniques serve for analyzing protein molecules; the recently introduced carbohydrate microarrays for the analysis of carbohydrate molecules and various cellomics assays for the analysis of integrated cells.

DNA microarrays or DNA chips comprise a plurality of DNA strands (probes or targets) immobilized on a surface of a substrate, where probes or targets of known identity are located in known and hence addressable locations over the surface of the substrate In a typical assay in which a DNA microarray is used in the analysis of nucleic acids, single stranded molecules (targets or probes, respectively), typically oligonucleotides or cDNA, tagged with fluorescent markers, are interacted with the substrate, resulting in hybridization of targets and probes according to the DNA parity rules. Following appropriate washes, the chip is scanned, typically with a laser-scanner, which excites the fluorescent tags (where present) and reads the emitted light. Depending on the application, the pattern of fluorescence over the surface of the chip provides information on the sequence of the targets and/or the expression level of a variety of genes.

The basic limitation associated with the use of nucleic acid microarrays is the ‘flood’ of poor quality data. Currently about 90% of the data is insignificant. In most cases, weakly expressed genes that can be very important in a biological pathway, are not detected. This limitation arises from the poor signal-to-noise ratio (S/N) and insufficient sensitivity of this technique. It further leads to poor reproducibility. It is difficult to quantify the result of an experiment. The results of seemingly identical experiments also vary considerably. Furthermore, in many cases, the genes of most importance produce a weak signal that is not at all detected.

Hence, there is a great need to increase the sensitivity and dynamic range of microarrays and reduce their inherent noise and background levels.

These challenges are possibly achievable by substantially miniaturizing the microarrays. The miniaturization is important since it will provide a possibility to imprint larger portions of the genome on the same array (perhaps even the entire human genome). An additional reason for miniaturization is the long period of time required for the target molecules to cover an array by diffusion. The smaller the array, the shorter this time is, in a quadratic manner. nevertheless, the presently employed analyzing techniques, i.e., the use of fluorescent tags and laser scanning, impose great limitations on further miniaturization, both with respect to spatial resolution and with respect to scanning time.

An additional type of limitations of the presently employed microarrays arise from the bleaching of the fluorescent tags once analyzed. After an array is scanned, it is bleached, meaning that the fluorescent tags emit less than the required intensity of light. This property significantly reduces the ability of a user to repeat a measurement of a pre-measured experiment.

Mainly due to the above limitations, there is no standard by which microarray experiments are performed and/or analyzed, leaving a too large room for personal know-how. In many cases, experiments executed in different laboratories cannot be repeated or even compared.

Hence, it is evident that there is a great need for a microarray detection system whose quantification is limited only by the biology. Preferably this system will be based on single molecule detection. This system should be free of the limitations associated with fluorescence-based readings and advantageously should have the following properties: (i) high sensitivity; (ii) high S/N ratio; (iii) compatibility with miniaturization of the microarray, and with smaller sample sizes; (iv) it should not bleach, providing the opportunity to rescan a sample or its regions of interest more than once; and (v) it should be able to incorporate assisted hybridization processes (not only diffusion).

One objective of the present invention is to disclose a microarray, scanning method and system, capable of performing high throughput detection on the level of a single molecule. This system is sensitive and reproducible enough to set the industry standard.

One option that may be considered is the use of a Scanning Force Microscope in the analysis of microarrays. However, the inherently low throughput of this system prevents it from being practical. The present invention solves the above mentioned limitations by means of scanning electron microscopy.

An additional limitation, complementary to quantifying genes in microarrays, is the quantitative study of proteomics (the study of proteins). Proteins determine many biological processes and are very important to drug discovery and many other applications.

One tool in proteomics is high resolution two-dimensional polyacrylamide gel electrophoresis (2D PAGE) and the image analysis thereof Electrophoresis is the migration of charged molecules in a solution, in response to an electric field. The rate of migration depends on the strength of the field, on the charge, size and shape of the molecules, as well as on the parameters of the medium through which the molecules are moving. 2D gel electrophoresis is a method to separate molecules that differ in any combination of size or charge. The solution is supported by a gel (agarose, polyacrylamide), which prevents undesired migration (convection, diffusion) and sieves the molecules, thus contributing to their separation on the basis of their sizes. In the present application, this system is referred to as 2D PAGE. The scope is not limited to any particular gel.

2D PAGE systems typically resolve about 1000 proteins according to their isoelectric propertied through a pH gradient in one direction and thereafter according to their size, in the presence of SDS, in a second direction, perpendicular to the first. The abundance of proteins in a cell is within a range from single to millions of molecules. There are many proteins in the gel that are not resolved, partly due to the lack of sensitivity of the separation and partly due to the lack of sensitivity of the detection.

There are two typical phases in protein analysis: (i) separation of the proteins, e.g., via 2D PAGE; and (ii) analyzing the types of the separated proteins, typically by mass spectrometry.

What is clearly missing is an intermediate stage where the number of proteins in each spot is counted. Preferably the counting method will be able to distinguish between the different proteins. Preferably it will rely on single molecule detection.

Currently there is no technology that provides a satisfactory quantitative answer to the issue of how many of the separated proteins exist in each spot. An additional question is how many types of proteins exist in each spot. Thus, there is a need for a technology that counts the number of proteins before they are inserted in the mass spectrometer.

The above-mentioned limitations of genomics and proteomics technologies are amplified in the emerging technology of protein microarrays.

Protein microarrays or protein chips comprise a plurality of proteins (probes or targets) immobilized on a surface of a substrate, where probes or targets of known identity are located in known and hence addressable locations over the surface of the substrate. In a typical assay in which a protein microarray is used in the analysis of proteins, protein molecules (targets or probes, respectively), typically antigens or antibodies, tagged with fluorescent markers, are interacted with the substrate, resulting in binding of targets and probes according to their identity. Following appropriate washes, the chip is scanned, typically with a laser-scanner, which excites the fluorescent tags (where present) and reads the emitted light. Depending on the application, the pattern of fluorescence over the surface of the chip provides information on the identity of the targets and/or the level of their expression. In many cases, comparative competition assays are performed, where a change in the pattern of fluorescence is indicative of the identity of the targets and/or the level of their expression.

Protein microarrays will go a long way towards elucidating aspects of cellular functions that DNA chips cannot provide, since measuring mRNA levels alone ignores issues which are of great influence on cellular function, such as, but not limited to, protein lifetime, protein post transnational modifications, etc. Protein chips find uses in two major fields: drug discovery and diagnostics. In drug discovery, processes such as drug candidate discovery and candidate optimization can be greatly assisted should highly sensitive and reliable protein chips and analysis methods were available. In diagnostics, determining titers of viruses and other pathogens, presence, absence or level of cancer and other markers, antibody profiles, etc., could be greatly assisted should highly sensitive and reliable protein chips and analysis methods were available.

It is apparent that proteomic tools are essential to obtain information that is unavailable when performing analysis on the gene level. Expressed genes can be subjected to significant post-translational regulation, and proteins undergo significant post-translational modifications (such as phosphorylation, acetylation, etc.) that significantly affect their function at the cellular level. In many cases, no correlation exists between the level of a specific messenger RNA and the level/activity of its encoded protein, because, most of the control of the expression of that protein takes place at the post translational phase. To this effect, see, for example, S. P. Gygi, et al., Mol. Cell. Biol. 19 (1999) 1720-1730).

Recently it was shown that it is possible to array different proteins, or protein ligands on a microscope slide to study a variety of protein functions (Macbeath et al., Nature 289, 2000). The basic challenge is that the proteins bound on surface retain their activity and/or their antigenic epitopes. The proteins attached covalently to the slide surface yet retained their ability to interact specifically with other macromolecules, e.g., other proteins, or with small molecules in solution. In this study the proteins attached to the slides were probed with fluorescently-labeled proteins. Screening for protein-protein interactions, substrates of protein kinases and targets of small molecules was demonstrated.

It often occurs that the less highly expressed proteins are those that are of most interest since their response to various physiological stimuli is the most interesting and informative. Unfortunately, it seems that the low abundance proteins, such as hormones, cytokines, small G-proteins, DNA binding proteins etc., are not easily detected by the present proteomics techniques (S. P. Gygi, Proc. Natl. Acad. Sci. USA 97 (2000) 9390-9395). Detection on the single or close to single molecule level usually requires painstaking techniques that require tedious sample preparation and imaging and are hard to apply to high throughput methods.

Carbohydrate microarrays comprise a plurality of carbohydrates (probes or targets) immobilized on a surface of a substrate, where probes or targets of known identity are located in known and hence addressable locations over the surface of the substrate. In a typical assay in which a carbohydrate microarray is used in the analysis of carbohydrates, molecules such as antibodies directly or indirectly tagged with fluorescent markers, are interacted with the substrate, resulting in binding of targets and probes according to their identity. Following appropriate washes, the chip is scanned, typically with a laser-scanner, which excites the fluorescent tags (where present) and reads the emitted light. Depending on the application, the pattern of fluorescence over the surface of the chip provides information on the identity of the targets/probes and/or the level of their expression. The limitations described hereinabove with respect to nucleic acid and protein microarrays clearly apply also to carbohydrate microarrays.

Cell microarrays comprise a plurality of cells immobilized on a surface of a substrate, which cells can be screened for various properties in a living or fixated state. The limitations described hereinabove with respect to nucleic acid and protein microarrays clearly apply also to carbohydrate microarrays.

There is thus a widely recognized need for, and it would be highly advantageous to have, a technique for the implementation of genomics, proteomics glycomics and cellomics devoid of the above limitations. In particular, it would be highly advantageous to have a technique for implementing genomics, proteomics glycomics or cellomics that reaches single molecule detection levels, yields high signal-to-noise ratios, and demonstrates a broad dynamic range, while, at the same time, retaining simple sample preparation, readily applicable for high throughput screening.

### SUMMARY OF THE INVENTION

It is an object of the present invention to provide a method for identification and quantification of biological molecules in a sample that overcomes the drawbacks of the existing methods

The term biological molecule includes any molecule with biological relevance. This includes, but is not limited to: polysaccharides, small chemical molecules such as lipids, peptides, hormones and other messengers, ATP GTP etc., drugs, non proteinaceous antigens and any homo- (e.g., protein-protein as example) and hetero- (e.g., drug-protein, DNA-RNA, DNA-protein, etc.) complexes, as well as chemically modifications and derivatisations whether naturally occurring or man made, of all these different molecules.

It is another object of the present invention to provide such a method for identification and quantification of biological molecules in a sample that is based on single-molecule detection, has a higher sensitivity and signal-to-noise ratio and broader dynamic range than existing methods.

It is a further object of the present invention to provide such a method for identification and quantification of biological molecules in a sample that is carried out without the bleaching that characterizes fluorescence-based detection, thus enabling measuring the same sample several times.

It is a further object of the present invention to provide such a method for identification and quantification of biological molecules in a sample that is carried out with an Environmental Scanning Electron Microscope (ESEM), thus enabling the investigation of a sample at almost atmospheric pressure.

It is a further object of the present invention to provide such a method for identification and quantification of biological molecules in a sample that is carried out with a Wafer Inspection Scanning Electron Microscope (WISEM), thus greatly reducing the cost of instrumentation required to implement the method.

It is still a further object of the present invention to provide such a method for identification and quantification of biological molecules in a sample that is carried out on a miniaturized microarray and allows to imprint larger portions of the human genome, or even the entire human genome, on the same array.

According to one aspect of the present invention there is provided a method of detecting binding between first member or members of a binding pair and corresponding second member or members of the binding pair, the method comprising interacting a solid support onto which the first member or members of the binding pair being immobilized and arrayed with the corresponding second member or members of the binding pair, the corresponding second member or members of the binding pair being directly or indirectly tagged with a heavy atom; and determining a spatial distribution of the heavy atom over a surface of the solid support, thereby detecting the binding between the first member or members of the binding pair and the corresponding second member or members of the binding pair. Preferably, determining the spatial distribution of the heavy atom over the surface of the solid support is at a dynamic range of linearity of at least four orders-of-magnitude. Still preferably, determining the spatial distribution of the heavy atom over the surface of the solid support is at a sensitivity of detection equals to or greater than 1 of 10 binding events, e.g., i of 5 binding events, most preferably, about 1 of 1 binding events. Yet preferably, determining the spatial distribution of the heavy atom over the surface of the solid support is at a signal-to-noise ratio greater than 20, preferably greater than 50, more preferably greater than 80.

According to another aspect of the present invention there is provided a method of detecting binding between first member or members of a binding pair and corresponding second member or members of the binding pair, the method comprising interacting a solid support onto which the first member or members of the binding pair being immobilized and arrayed with the corresponding second member or members of the binding pair; and determining a spatial distribution of the second member or members of the binding pair at a dynamic range of linearity of at least four orders-of-magnitude. Preferably, the corresponding second member or members of the binding pair are directly or indirectly tagged with a heavy atom, whereas determining the spatial distribution of the second member or members of the binding pair is by determining a spatial distribution of the heavy atom over the surface of the solid support. Still preferably, determining the spatial distribution of the heavy atom over the surface of the solid support is at a dynamic range of linearity of at least four orders-of-magnitude. Yet preferably, determining the spatial distribution of the second member or members of the binding pair over the surface of the solid support is at a sensitivity of detection equals to or greater than I of 10 binding events, e.g., equals to or greater than I of 5 binding events, optimally the sensitivity is about 1 of 1 binding events. Still preferably, determining the spatial distribution of the second member or members of the binding pair over the surface of the solid support is at a signal-to-noise ratio greater than 20, preferably greater than 50, more preferably, greater than 80.

According to still another aspect of the present invention there is provided a method of detecting binding between first member or members of a binding pair and corresponding second member or members of the binding pair, the method comprising interacting a solid support onto which the first member or members of the binding pair being immobilized and arrayed with the corresponding second member or members of the binding pair; and determining a spatial distribution of the second member or members of the binding pair at a sensitivity of detection equals to or greater than 1 of 10 binding events, preferably, the sensitivity equals to or greater than 1 of 5 binding events, more preferably, the sensitivity equals to about 1 of 1 binding events. In a preferred embodiment, the corresponding second member or members of the binding pair are directly or indirectly tagged with a heavy atom, whereas determining the spatial distribution of the second member or members of the binding pair is by determining a spatial distribution of the heavy atom over the surface of the solid support. Preferably, determining the spatial distribution of the second member or members of the binding pair over the surface of the solid support is at a dynamic range of linearity of at least four orders-of-magnitude. Still preferably, determining the spatial distribution of the second member or members of the binding pair over the surface of the solid support is at a signal-to-noise ratio greater than 20, preferably, greater than 50, more preferably, greater than 80.

According to yet another aspect of the present invention there is provided a method of detecting binding between first member or members of a binding pair and corresponding second member or members of the binding pair, the method comprising interacting a solid support onto which the first member or members of the binding pair being immobilized and arrayed with the corresponding second member or members of the binding pair; and determining a spatial distribution of the second member or members of the binding pair at a signal-to-noise ratio greater than 20, preferably, greater than 50, more preferably, greater than 80. Preferably, the corresponding second member or members of the binding pair are directly or indirectly tagged with a heavy atom, whereas determining the spatial distribution of the second member or members of the binding pair is by determining a spatial distribution of the heavy atom over the surface of the solid support. Still preferably, determining the spatial distribution of the second member or members of the binding pair over the surface of the solid support is at a dynamic range of linearity of at least four orders-of-magnitude. Yet preferably, determining the spatial distribution of the second member or members of the binding pair over the surface of the solid support is at a sensitivity of detection equals to or greater than 1 of 10 binding events, preferably, the sensitivity equals to or greater than 1 of 5 binding events, most preferably, the sensitivity is about 1 of 1 binding events.

According to further features in preferred embodiments of the invention described below, the binding pair is selected from the group consisting of antigen-antibody, antibody-antigen, hapten-antibody, antibody-hapten, nucleic acid-complementary nucleic acid, nucleic acid-substantially complementary nucleic acid, ligand-receptor, receptor-ligand, enzyme-substrate, substrate-enzyme, enzyme-inhibitor and inhibitor-enzyme.

According to still further features in the described preferred embodiments determining the spatial distribution of the heavy atom over the surface of the solid support is by particle scattering. Preferably, determining the spatial distribution of the heavy atom over the surface of the solid support is by electron scattering.

According to still further features in the described preferred embodiments the corresponding second member or members of the binding pair is indirectly tagged with a heavy atom.

According to still further features in the described preferred embodiments the heavy atom is selected from the group consisting of gold, silver and iron.

According to an additional aspect of the present invention there is provided a method of identifying and/or quantifying at least one biological molecule in a sample, the method comprising contacting the sample with a microarray presenting an addressable array of macromolecules of known identities under conditions so as to allow binding between the at least one biological molecule and the macromolecules of known identities; detecting a spatial distribution of the at least one biological molecule over a surface of the microarray at a dynamic range of linearity of at least four orders-of-magnitude, thereby identifying and/or quantifying the least one biological molecule in the sample. Preferably, detecting the spatial distribution of the at least one biological molecule over the surface of the microarray is at a sensitivity equals to or greater than 1 of 10 binding events. Still preferably, detecting the spatial distribution of the at least one biological molecule over the surface of the microarray is at a signal-to-noise ratio greater than 20. Yet preferably, detecting the spatial distribution of the at least one biological molecule over the surface of the microarray is by directly or indirectly tagging the at least one biological molecule with at least one heavy atom and obtaining a particle scattering image of a spatial distribution of the at least one heavy atom.

According to still an additional aspect of the present invention there is provided a method of identifying and/or quantifying at least one biological molecule in a sample, the method comprising contacting the sample with a microarray presenting an addressable array of macromolecules of known identities under conditions so as to allow binding between the at least one biological molecule and the macromolecules of known identities; and detecting a spatial distribution of the at least one biological molecule over a surface of the microarray at a sensitivity equals to or greater than 1 of 10 binding events, thereby identifying and/or quantifying the least one biological molecule in the sample. Preferably, detecting the spatial distribution of the at least one biological molecule over the surface of the microarray is at a dynamic range of linearity of at least four orders-of-magnitude. Still preferably, detecting the spatial distribution of the at least one biological molecule over the surface of the microarray is at a signal-to-noise ratio greater than 20. Yet preferably, detecting the spatial distribution of the at least one biological molecule over the surface of the microarray is by directly or indirectly tagging the at least one biological molecule with at least one heavy atom and obtaining a particle scattering image of a spatial distribution of the at least one heavy atom.

According to yet an additional aspect of the present invention there is provided a method of identifying and/or quantifying at least one biological molecule in a sample, the method comprising contacting the sample with a microarray presenting an addressable array of macromolecules of known identities under conditions so as to allow binding between the at least one biological molecule and the macromolecules of known identities; and detecting a spatial distribution of the at least one biological molecule over a surface of the microarray at a signal-to-noise ratio greater than 20, thereby identifying and/or quantifying the least one biological molecule in the sample. Preferably, detecting the spatial distribution of the at least one biological molecule over the surface of the microarray is at a dynamic range of linearity of at least four orders-of-magnitude. Still preferably, detecting the spatial distribution of the at least one biological molecule over the surface of the microarray is at a sensitivity equals to or greater than 1 of 10 binding events. Yet preferably, detecting the spatial distribution of the at least one biological molecule over the surface of the microarray is by directly or indirectly tagging the at least one biological molecule with at least one heavy atom and obtaining a particle scattering image of a spatial distribution of the at least one heavy atom.

According to still an additional aspect of the present invention there is provided a method of identifying and/or quantifying at least one biological molecule in a sample, the method comprising contacting the sample with a microarray presenting an addressable array of macromolecules of known identities under conditions so as to allow binding between the at least one biological molecule and the macromolecules of known identities; and detecting a spatial distribution of the at least one biological molecule over a surface of the microarray by directly or indirectly tagging the at least one biological molecule with at least one heavy atom and obtaining a particle scattering image of a spatial distribution of the at least one heavy atom, thereby identifying and/or quantifying the least one biological molecule in the sample. Preferably, detecting the spatial distribution of the at least one biological molecule over the surface of the microarray is at a dynamic range of linearity of at least four orders-of-magnitude. Still preferably, detecting the spatial distribution of the at least one biological molecule over the surface of the microarray is at a sensitivity equals to or greater than 1 of 10 binding events. Yet preferably, detecting the spatial distribution of the at least one biological molecule over the surface of the microarray is at a signal-to-noise ratio greater than 20.

According to further features in preferred embodiments of the invention described below, the at least one biological molecule is selected from the group consisting of a protein, a glycoprotein, a nucleic acid and a carbohydrate.

According to still further features in the described preferred embodiments the macromolecules of known identities are selected from the group consisting of proteins, glycoproteins, nucleic acids and carbohydrates.

According to another aspect of the present invention there is provided a method of identifying and/or quantifying at least one biological molecule in a sample, the method comprising attaching biological molecules present in the sample to a solid support; contacting the solid support with at least one macromolecule of a known identity under conditions so as to allow binding between the at least one biological molecule and the at least one macromolecule of known identity; and detecting a level of binding between the at least one biological molecule and the at least one macromolecule of known identity at a dynamic range of linearity of at least four orders-of-magnitude, thereby identifying and/or quantifying the least one biological molecule in the sample. Preferably, detecting the level of binding between the at least one biological molecule and the at least one macromolecule of known identity is at a sensitivity equals to or greater than 1 of 10 binding events. Still preferably, detecting a level of binding between the at least one biological molecule and the at least one macromolecule of known identity is at a signal-to-noise ratio greater than 20. Yet preferably, detecting a level of binding between the at least one biological molecule and the at least one macromolecule of known identity is by directly or indirectly tagging the at least one macromolecule of known identity with at least one heavy atom and obtaining a particle scattering image of a spatial distribution of the at least one heavy atom.

According to still another aspect of the present invention there is provided a method of identifying and/or quantifying at least one biological molecule in a sample, the method comprising attaching biological molecules present in the sample to a solid support; contacting the solid support with at least one macromolecule of a known identity under conditions so as to allow binding between the at least one biological molecule and the at least one macromolecule of known identity; and detecting a level of binding between the at least one biological molecule and the at least one macromolecule of known identity at a sensitivity equals to or greater than 1 of 10 binding events, thereby identifying and/or quantifying the least one biological molecule in the sample. Preferably, detecting the level of binding between the at least one biological molecule and the at least one macromolecule of known identity is at a dynamic range of linearity of at least four orders-of-magnitude. Still preferably, detecting a level of binding between the at least one biological molecule and the at least one macromolecule of known identity is at a signal-to-noise ratio greater than 20. Yet preferably, detecting a level of binding between the at least one biological molecule and the at least one macromolecule of known identity is by directly or indirectly tagging the at least one macromolecule of known identity with at least one heavy atom and obtaining a particle scattering image of a spatial distribution of the at least one heavy atom.

According to a further aspect of the present invention there is provided a method of identifying and/or quantifying at least one biological molecule in a sample, the method comprising attaching biological molecules present in the sample to a solid support; contacting the solid support with at least one macromolecule of a known identity under conditions so as to allow binding between the at least one biological molecule and the at least one macromolecule of known identity; and detecting a level of binding between the at least one biological molecule and the at least one macromolecule of known identity at a signal-to-noise ratio greater than 20, thereby identifying and/or quantifying the least one biological molecule in the sample. Preferably, detecting the level of binding between the at least one biological molecule and the at least one macromolecule of known identity is at a dynamic range of linearity of at least four orders-of-magnitude. Still preferably, detecting a level of binding between the at least one biological molecule and the at least one macromolecule of known identity is at a sensitivity greater than or equals to 1 of 10 binding events. Yet preferably, detecting a level of binding between the at least one biological molecule and the at least one macromolecule of known identity is by directly or indirectly tagging the at least one macromolecule of known identity with at least one heavy atom and obtaining a particle scattering image of a spatial distribution of the at least one heavy atom.

According to still a further aspect of the present invention there is provided a method of identifying and/or quantifying at least one biological molecule in a sample, the method comprising attaching biological molecules present in the sample to a solid support; contacting the solid support with at least one macromolecule of a known identity under conditions so as to allow binding between the at least one biological molecule and the at least one macromolecule of known identity; and detecting a level of binding between the at least one biological molecule and the at least one macromolecule of known identity by directly or indirectly tagging the at least one macromolecule of known identity with at least one heavy atom and obtaining a particle scattering image of a spatial distribution of the at least one heavy atom, thereby identifying and/or quantifying the least one biological molecule in the sample. Preferably, detecting the level of binding between the at least one biological molecule and the at least one macromolecule of known identity is at a dynamic range of linearity of at least four orders-of-magnitude. Still preferably, detecting a level of binding between the at least one biological molecule and the at least one macromolecule of known identity is at a sensitivity greater than or equals to 1 of 10 binding events. Yet preferably, detecting a level of binding between the at least one biological molecule and the at least one macromolecule of known identity is at a signal-to-noise ratio greater than 20.

According to further features in preferred embodiments of the invention described below, the at least one biological molecule is selected from the group consisting of a protein, a glycoprotein, a nucleic acid and a carbohydrate.

According to still further features in the described preferred embodiments the at least one macromolecule of known identity is selected from the group consisting of a protein, a glycoprotein, a nucleic acid and a carbohydrate.

According to another aspect of the present invention there is provided a method of identifying and/or quantifying biological molecules in a preparate, the method comprising localizing and tagging the biological molecules in the preparate; preparing the preparate for vacuum; loading the preparate into the specimen chamber of an electron beam device; irradiating the preparate with an electron beam, thus obtaining an image of the tags; and analyzing the image to quantity the biological molecules by image analysis software.

According to another aspect of the present invention there is provided a method of identifying and/or quantifying biological molecules in a preparate, the method comprising localizing and tagging the biological molecules in the preparate; loading the preparate into the specimen chamber of an electron beam device; irradiating the preparate with an electron beam, thus obtaining an image of the tags; and analyzing the image to quantify the biological molecules by image analysis software.

According to still another aspect of the present invention there is provided a method of identifying and/or quantifying biological molecules in a preparate, the method comprising localizing the biological molecules in the preparate; loading the preparate into the specimen chamber of an electron beam device; irradiating the preparate with an electron beam, thus obtaining an image representing the biological molecules; and analyzing the image to quantify the biological molecules by image analysis software.

According to still another aspect of the present invention there is provided an apparatus for inspection of a preparate of biological molecules comprising an electron source to provide an electron beam; a charged particle beam column to deliver and scan an electron beam from the electron source on the surface of the preparate; a vacuum system including a first and a second chamber in each of which pressurization can be performed independently to permit loading or unloading of a first preparate in one chamber while simultaneously inspecting a second preparate; at least one electron detector; means for measuring X-ray spectrum; a continuously moving x-y stage disposed to receive the preparate and to provide at least one degree of motion to the preparate while the preparate is being scanned; and means for carrying out image analysis of the molecules on the preparate.

The biological molecules may be polynucleotides, e.g. DNA, cDNA, RNA, clusters, or proteins such as antigens, antibodies. The term biological molecules also refers but is not limited to: polysaccharides, small chemical molecules such as lipids, peptides, hormones and other messengers, ATP antibodies, GTP, etc., drugs, non proteinaceous antigens and any homo-(protein-protein as example) and hetero- (drug-protein, DNA-RNA, DNA-protein etc.) complexes as well as chemically modifications and derivatisations whether naturally occurring or not of all these different molecules. The preparate for the polynucleotides may be a microarray, e.g., a DNA chip, and for the proteins may be a 2D PAGE, a protein chip, e.g., an antigen or antibody chip, cell chip, cell preparate, and the like.

The localization of the biological molecules may be carried out before or after tagging, depending on the type of the biological molecule and of the technique used.

When the biological molecule in the preparate is a polynucleotide, the localization may be carried out, for example, by hybridization, either to a polynucleotide of known sequence (probe) when the polynucleotide immobilized in the microarray preparate is of unknown sequence (target), or to a polynucleotide of unknown sequence (target) when the polynucleotide immobilized in the microarray preparate is of a known sequence (probe). The same is true for protein microarrays, with respect to either antigens and/or antibodies, each of which can serve as a target or probe, and in any case can be immobilized to the microarray or be interacted therewith.

When the biological molecule in the preparate is a protein, the localization may be carried out, for example, by separating the molecules by one- or two-dimensional electrophoresis, or by attaching the molecules to a blot membrane. When the preparate is a 2D PAGE or proteins extracted therefrom, the separation in the gel may be preferably performed on-line under the scanning electron beam. Identification of the proteins can be done by mass spectrometry.

The localization in space may further consist of localizing the molecules by their affiliation to specific biological cells.

Tagging of the biological molecules such as DNA, RNA and proteins, may be carried out with heavy metals such as silver or gold, for example using colloidal gold or gold clusters, or doping with metal-enriched organic compounds, wherein the metal is, for example, Fe. The heavy metal colloids (e.g., gold), preferably of diameter range of 1-200 nm, more preferably, less than 20 nm, create a high intensity back scattered electron signal and, therefore, high image contrast. In one embodiment, there is one tag per target molecule. More specifically the tagging may be carried out with biotin followed by gold tagged avidin.

Tagging may also be made with electro-luminescent molecules whereby the electron beam creates a light signal that is detected. Tagging may also be done with more than one type of tags to make a distinction between two preparates.

According to one embodiment multi-labeling or Multi-tagging is achieved. This is achieved, for example, by using gold colloids of a plurality of sizes. According to another embodiment, multi-labeling is achieved by using a combination of gold colloids and fluorescent labels. According to a yet another embodiment, the multi-labeling is achieved by using a plurality of metals that are read by the X-RAY reading apparatus of the SEM, such as Energy Depressive Spectrum and so forth.

According to one embodiment, the DNA molecules are not tagged and the SEM is sensitive enough to detect density differences between hybridized and non-hybridized regions. Direct detection with no tagging enables the identification of an additional variety of substances such as viral particles.

The preparates are prepared for vacuum by known standard methods that include drying, fixation and coating with a conductive layer such as carbon, to prevent charge accumulation., protection with a membrane and freezing to prevent out-gassing.

The preparates are examined in a particles beam device, preferably, an electron beam device, namely an electron microscope such as a scanning electron microscope (SEM). Presently, most preferably, the preparates are scanned and are analyzed using a wafer inspection SEM (WISEM) typically used in the microelectronics industry. The irradiation of the preparate is carried out in such a way as to form sufficient contrast of the electrons that are back scattered from the tags in comparison with those that are emitted/scattered from the background.

In another embodiment, the SEM system is an environmental scanning electron microscope (ESEM) that works at almost atmospheric pressure, thus minimizing the need to prepare the preparate for vacuum.

According to a yet another embodiment, the SEM system that allows the proteins to remain in their native wet state and still imaged. This embodiment utilizes a device and method that uses membrane partition. To this end, see U.S. Provisional Patent Application No. 60/250,879, which is incorporated herein by reference.

The image analysis may comprise any one of: performing edge detection algorithm to identify the colloids in each region-of-interest (ROI) and counting the colloids; counting fluorescence signals; and identifying X-ray spectrum of each particle for identification by comparison to a reference spectrum. The invention further relates to an apparatus for inspection of a preparate of biological molecules according to the above method, the apparatus comprising an electron source to provide an electron beam; a charged particle beam column to deliver and scan an electron beam from the electron source on the surface of the preparate; a vacuum system including a first and a second chamber in each of which pressurization can be performed independently to permit loading or unloading of a first preparate in one chamber while simultaneously inspecting a second preparate; at least one electron detector; means for measuring X-ray spectrum; a continuously moving x-y stage disposed to receive the preparate and to provide at least one degree of motion to the preparate while the preparate is being scanned by the electron beam; and means for carrying out image analysis of the molecules on the preparate.

In one preferred embodiment, the charged particle beam column of is a microcolumn.

In a further embodiment, the invention provides a method for the inspection of biological molecules on a preparate using an electron beam, the method comprising localizing the biological molecules in space and tagging them with markers; preparing the preparate for vacuum; taking out the preparate to be analyzed from the preparate cassette; pre-aligning preparate and read preparate number; reading a recipe that contains the information to be detected; loading the preparate on X-Y-T stage (T means tilt) of an electron beam device; aligning the preparate; moving XYT stage to analysis position; positioning the electron beam on the substrate accurately by measuring the position of the substrate; scanning the preparate at low resolution to create a preparate map, while enhancing contrast; determining the regions-of-interest (ROI) spots on the map that should be scanned in a high resolution; scanning the ROIs with the electron beam as the substrate is continuously moving with at least one degree of motion in an x-y plane; detecting electrons emanating from the substrate as a result of previous step and forming an image; enhancing the image contrast; storing both modified and bare image; analyzing the ROIs; and displaying the results.

The present invention successfully addresses the shortcomings of the presently known configurations by providing a technique for the implementation of genomics, proteomics, glycomics and cellomics that reaches the highest sensitivity of ultimately single molecule detection, yields high signal-to-noise ratios, and demonstrates a broad dynamic range, while, at the same time, retaining simple preparate preparation, readily applicable for high throughput screening.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a flow-chart illustrating steps of a method according to the teachings of the present invention.

FIG. 2 is a general diagram showing a longitudinal cross-section of an SEM used as a preparate analysis apparatus according to the present invention.

FIG. 3 is a generalized diagram showing a cross-section of a scanning electron microscope according to an aspect of the present invention.

FIG. 4 shows an apparatus that combines an ESEM with a SEM according to the present invention.

FIG. 5 presents another embodiment of the present invention, whereby the detection is made by exciting light photons (electro-luminescence).

FIG. 6 a SEM device having a microcolumns array for use in the method of the present invention.

FIG. 7 is a gel chamber for use in the method of the present invention.

FIG. 8 demonstrates tagged proteins immobilized on a surface according to the present invention.

FIG. 9 is an image showing gold conjugate proteins as imaged in SEM without image processing according to the present invention.

FIG. 10 is a bar graph and a table demonstrating signal (S) to background (B) ratios (defined as (S-B)/B) for STP20, STP40 and Cy3-STP probes in the BSA-biotin—gold/Cy3-streptavidin detection system. Only last five dilutions of BSA-biotin are shown.

FIG. 11 is a blow-up of FIG. 10, where only last three dilutions of BSA-biotin are shown.

FIG. 12 is a bar graph demonstrating signal (S) to background (B) ratios (defined as (S-B)/B) for STP20 and Cy3-STP probes. The data for Cy3-STP probes was obtained by averaging over results of 4 independent slides.

FIG. 13 is a graph demonstrating the estimated detection abilities presented as the number of biotinylated BSA molecules detected with STP20 probe versus the total number of biotinylated BSA molecules present in a spot on the slide. The number of biotinylated BSA molecules conjugated to the slide is approximated by calculating the number of molecules being able to attach to the surface. As shown in the calculations below, this is at most 10% of the total molecules contained in a 10 ml drop that was spotted on the slide. This is the upper limit to this number, since most likely less then 10% of the molecules contained in the drop indeed conjugated to the glass surface. The number of molecules detected is given by the number of gold colloids detected. This number was calculated by extrapolating from the average number of gold colloids counted in a single SEM frame (see, FIG. 14) to the area of the whole drop. The dashed line represents a linear fit. From the slope of the fit and the efficiency of attachment of biotinylated BSA to the slide it arises that the detection ability if of between 1:1 and 1:4, hence, the upper limit of detection (sensitivity) was reached, over an unprecedented dynamic range.

FIG. 14 is a backscattered electrons image demonstrating single molecule detection using 20 nm gold colloids according to the present invention. The high quality is achieved using backscattered electrons in accordance with the teachings of the present invention. The number of gold colloids can easily and accurately be quantified manually or via using a simple image analysis software.

FIG. 15 is a graph demonstrating signal (S) to background (noise, B) ratios (defined as (S-B)/B) for STP40 probe in the BSA-hapten—biotinylated antibody—gold streptavidin detection system of the present invention employing different dilutions of the biotinylated antibody.

### DESCRIPTION OF THE PREFERRED EMBODIMENTS

The present invention relates to a method and apparatus useful in the implementation of genomics, proteomics, glycomics and cellomics. In particular, the method and apparatus of the present invention allows highest sensitivity of ultimately single molecule detection, yields high signal-to-noise ratios, and demonstrates a broad dynamic range, while, at the same time, retaining simple sample preparation, readily applicable for high throughput screening.

According to one aspect of the present invention there is provided a method of detecting binding between first member or members of a binding pair and corresponding second member or members of the binding pair. The method according to this aspect of the invention is implemented by interacting a solid support onto which the first member or members of the binding pair are immobilized and arrayed with the corresponding second member or members of the binding pair. The corresponding second member or members of the binding pair are directly or indirectly tagged with a heavy atom. Thereafter, a spatial distribution of the heavy atom over a surface of the solid support is determined, thereby binding between the first member or members of the binding pair and the corresponding second member or members of the binding pair is detected.

According to another aspect of the present invention there is provided a method of detecting binding between first member or members of a binding pair and corresponding second member or members of the binding pair. The method according to this aspect of the invention is implemented by interacting a solid support onto which the first member or members of the binding pair is immobilized and arrayed with the corresponding second member or members of the binding pair. Thereafter, the spatial distribution of the second member or members of the binding pair is determined at a dynamic range of linearity of at least four, preferably at least five, more preferably at least six, still preferably at least seven or at least eight orders-of-magnitude.

According to still another aspect of the present invention there is provided a method of detecting binding between first member or members of a binding pair and corresponding second member or members of the binding pair. The method according to this aspect of the invention is implemented by interacting a solid support onto which the first member or members of the binding pair are immobilized and arrayed with the corresponding second member or members of the binding pair. Thereafter, the spatial distribution of the second member or members of the binding pair is determined at a sensitivity of detection which equals to or is greater than 1 of 10 binding events, preferably, the sensitivity equals to or greater than 1 of 5 binding events, more preferably, the sensitivity equals to about 1 of 1 binding events.

According to yet another aspect of the present invention there is provided a method of detecting binding between first member or members of a binding pair and corresponding second member or members of the binding pair. The method according to this aspect of the invention is implemented by interacting a solid support onto which the first member or members of the binding pair are immobilized and arrayed with the corresponding second member or members of the binding pair. Thereafter, the spatial distribution of the second member or members of the binding pair at a signal-to-noise ratio greater than 20, preferably, greater than 50, more preferably, greater than 80, still preferably greater than 100.

The first and second members of the binding pair according to the present invention can be of any biochemical or chemical nature and serve any physiological or therapeutic function. First and second members of binding pair according to the present invention, include, for example, antigen-antibody, antibody-antigen, hapten-antibody, antibody-hapten, nucleic acid-complementary nucleic acid, nucleic acid-substantially complementary nucleic acid, ligand-receptor, receptor-ligand, enzyme-substrate, substrate-enzyme, enzyme-inhibitor and inhibitor-enzyme.

As used herein, the term “antigen” includes molecules having at least one epitope recognized by an antibody. Such a molecule can be, for example, a protein or a part thereof, a carbohydrate or a part thereof or any natural or man made chemical.

As used herein, the term “hapten” relates to a molecule or a portion of a macromolecule to which an antibody may specifically bind.

As used herein, the term “antibody”, includes polyclonal antibody, monoclonal antibody, fragment of an antibody, single chain antibody and a chimeric antibody. The source of the antibody can be from the serum of an immuned animal, a serum of a patient or produced by immortalized cells, such as hybridomas or virus infected antibody producing cells.

As used herein, the term “nucleic acid” includes natural nucleic acids such as DNA and RNA, either derived from nature or synthetically prepared, as well as analog nucleic acids capable of base pairing with natural nucleic acids.

As used herein, the phrase “complementary nucleic acid” refers to a nucleic acid as this term is defined above having a sequence of nucleobases, each of which matches a corresponding nucleobase in another nucleic acid according to the base parity rules.

As used herein, the phrase “substantially complementary nucleic acid” refers to a nucleic acid having a sequence of nucleobases, most of which (e.g., above 50%, preferably above 60%, more preferably, above 70%, still preferably above 80%, yet preferably, above 90%) match corresponding nucleobases in another nucleic acid according to the base parity rules.

As used herein the term “ligand” includes natural or man made molecules or macromolecules which are capable of binding to a receptor. A ligand can be, for example, a protein, a nucleic acid, a carbohydrate or a small molecule, including for example lipids, steroids, etc. The ligand can act as an agonist or antagonist when it binds the receptor. As such, a ligand can be a drug, a hormone, etc.

As used herein the term “receptor” includes macromolecules that bind ligands. Such macromolecules may for example be proteinaceous, soluble or anchored to a membrane.

As used herein the term “enzyme” refers to a proteinaceous macromolecule having catalytic activity with respect to one or more substrates.

As used herein the term “substrates” refers to any kind of molecule which undergoes faster catalysis in the presence of an enzyme.

As used herein the term “inhibitor” includes any molecule capable of reversibly or irreversibly slow down catalysis. As such, an inhibitor can be a drug.

According to the present invention determining the spatial distribution of a heavy atom over the surface of the solid support is by particle scattering. Preferably, determining the spatial distribution of the heavy atom over the surface of the solid support is by electron scattering. Any and all devices capable of producing a particle beam and recording scattered particles are suitable for implementing the present invention. Examples of such devices are described in more detail hereinafter.

The corresponding second member or members of the binding pair used in accordance with the present invention can be either directly or indirectly tagged with a heavy atom. Preferably, the corresponding second member or members of the binding pair used in accordance with the present invention is indirectly tagged with a heavy atom. It will be appreciated by one of skills in the art that indirectly tagging the corresponding second member or members of the binding pair provides an element of universality to the method of the present invention. The heavy atom can be any atom capable of scattering particles better than the atoms making organic molecules, such as C, H, O, N, S and P. Suitable heavy atoms include gold, silver and iron, which are frequently used in electron microscopy. Other heavy atoms, such as osmium and platinum may also be considered. Methods are known to link such heavy atoms to a variety of organic molecules. For example, gold can covalently bind through an SH group which is natural to proteins and can be introduced in other molecules such as nucleic acids and polysaccharides. Other heavy atoms can be trapped in suitable chelators, which can be linked to a variety of macromolecules using methods well known in the art.

According to an additional aspect of the present invention there is provided a method of identifying and/or quantifying at least one biological molecule in a sample. The method according to this aspect of the invention is implemented by contacting the sample with a microarray presenting an addressable array of macromolecules of known identities under conditions so as to allow binding between the at least one biological molecule and the macromolecules of known identities Thereafter, the spatial distribution of the at least one biological molecule over a surface of the microarray is determined at a dynamic range of linearity of at least four, preferably at least five, more preferably at least six, still preferably at least seven or at least eight orders-of-magnitude, thereby identifying and/or quantifying the least one biological molecule in the sample.

According to still an additional aspect of the present invention there is provided a method of identifying and/or quantifying at least one biological molecule in a sample. The method according to this aspect of the invention is implemented by contacting the sample with a microarray presenting an addressable array of macromolecules of known identities under conditions so as to allow binding between the at least one biological molecule and the macromolecules of known identities. Thereafter the spatial distribution of the at least one biological molecule over a surface of the microarray is detected at a sensitivity which equals to or is greater than 1 of 10 binding events, preferably, equals to or is greater than 1 of 5 binding events, more preferably, equals to about 1 of 1 binding events, thereby identifying and/or quantifying the least one biological molecule in the sample.

According to yet an additional aspect of the present invention there is provided a method of identifying and/or quantifying at least one biological molecule in a sample. The method according to this aspect of the invention is implemented by contacting the sample with a microarray presenting an addressable array of macromolecules of known identities under conditions so as to allow binding between the at least one biological molecule and the macromolecules of known identities. Thereafter the spatial distribution of the at least one biological molecule over a surface of the microarray is detected at a signal-to-noise ratio greater than 20, preferably, greater than 50, more preferably, greater than 80, still preferably greater than 100, thereby identifying and/or quantifying the least one biological molecule in the sample.

According to still an additional aspect of the present invention there is provided a method of identifying and/or quantifying at least one biological molecule in a sample. The method according to this aspect of the invention is implemented by contacting the sample with a microarray presenting an addressable array of macromolecules of known identities under conditions so as to allow binding between the at least one biological molecule and the macromolecules of known identities. Thereafter, the spatial distribution of the at least one biological molecule over a surface of the microarray is detected by directly or indirectly tagging the at least one biological molecule with at least one heavy atom and obtaining a particle scattering image of a spatial distribution of the at least one heavy atom, thereby identifying and/or quantifying the least one biological molecule in the sample.

According to another aspect of the present invention there is provided a method of identifying and/or quantifying at least one biological molecule in a sample. The method according to this aspect of the invention is implemented by attaching biological molecules present in the sample to a solid support; contacting the solid support with at least one macromolecule of a known identity under conditions so as to allow binding between the at least one biological molecule and the at least one macromolecule of known identity. Thereafter, the level of binding between the at least one biological molecule and the at least one macromolecule of known identity is determined at a dynamic range of linearity of at least four, preferably at least five, more preferably at least six, still preferably at least seven or at least eight orders-of-magnitude, thereby identifying and/or quantifying the least one biological molecule in the sample.

According to still another aspect of the present invention there is provided a method of identifying and/or quantifying at least one biological molecule in a sample. The method according to this aspect of the invention is implemented by attaching biological molecules present in the sample to a solid support; contacting the solid support with at least one macromolecule of a known identity under conditions so as to allow binding between the at least one biological molecule and the at least one macromolecule of known identity. Thereafter the level of binding between the at least one biological molecule and the at least one macromolecule of known identity is detected at a sensitivity a sensitivity which equals to or is greater than 1 of 10 binding events, preferably, equals to or is greater than 1 of 5 binding events, more preferably, equals to about 1 of 1 binding events, thereby identifying and/or quantifying the least one biological molecule in the sample.

According to a further aspect of the present invention there is provided a method of identifying and/or quantifying at least one biological molecule in a sample. The method according to this aspect of the invention is implemented by attaching biological molecules present in the sample to a solid support; contacting the solid support with at least one macromolecule of a known identity under conditions so as to allow binding between the at least one biological molecule and the at least one macromolecule of known identity. Thereafter, the level of binding between the at least one biological molecule and the at least one macromolecule of known identity is detected at a signal-to-noise ratio greater than 20, preferably, greater than 50, more preferably, greater than 80, still preferably greater than 100, thereby identifying and/or quantifying the least one biological molecule in the sample.

According to still a further aspect of the present invention there is provided a method of identifying and/or quantifying at least one biological molecule in a sample. The method according to this aspect of the invention is implemented by attaching biological molecules present in the sample to a solid support; contacting the solid support with at least one macromolecule of a known identity under conditions so as to allow binding between the at least one biological molecule and the at least one macromolecule of known identity. Thereafter the level of binding between the at least one biological molecule and the at least one macromolecule of known identity is detected by directly or indirectly tagging the at least one macromolecule of known identity with at least one heavy atom and obtaining a particle scattering image of a spatial distribution of the at least one heavy atom, thereby identifying and/or quantifying the least one biological molecule in the sample.

A method for the identification and quantification of biological molecules on a preparate such as a microarray or a 2D-gel according to preferred embodiments of the present invention includes the following steps (i) localization (the biological molecules are separated in space); (ii) tagging with markers (this step occurs before or after the localization, depending on the tagging, separation technology and/or preparate); (iii) scanning the tags; (iv) identifying or quantifying the tags; (iv) interpreting the number of molecules in each location from the quantity of tags and relating the results to the biological problem that is studied. According to the present invention, it is preferable to perform step (iii) with a scanning electron microscope (using appropriate tags in step (ii)). This provides a detection method that operates at high sensitivity, broad dynamic range and low background.

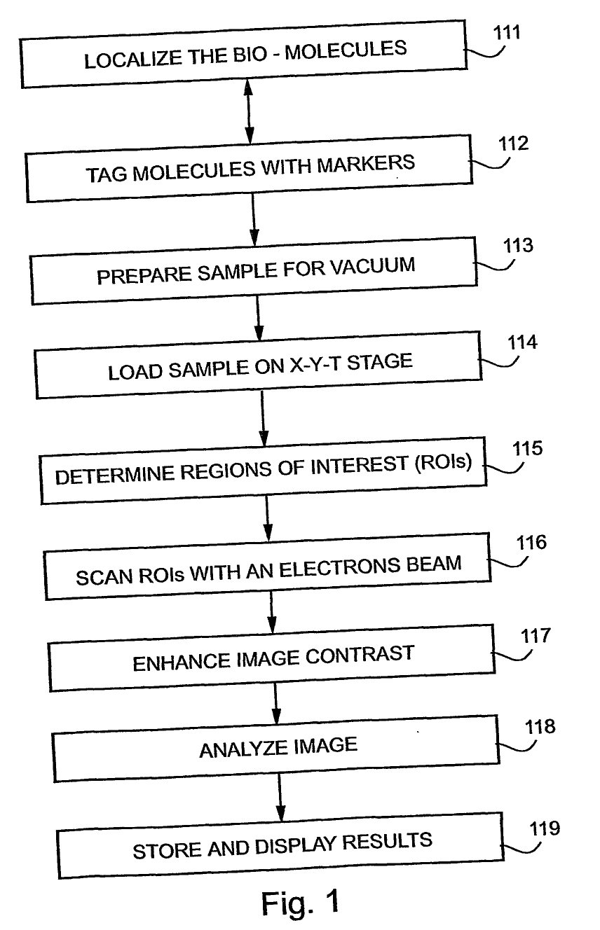

In one embodiment of the invention, the method is carried out as illustrated schematically in the flow-chart of FIG. 1, and comprises the following steps (1)-(9) (marked as 111-119 in FIG. 1):
1. Localizing the biomolecules in space.
2. Tagging the biomolecules with markers.
3. Preparing the preparate for vacuum.
4. Loading the preparate on X-Y-T stage (T means tilt) to analysis position.
5. Scanning the preparate at low resolution to identify the regions of interest (ROIs).
6. Scanning the ROIs with an electron beam at a high resolution.
7. Enhancing the image contrast.
8. Analyzing the ROIs.
9. Displaying results.

The step of localizing the biomolecules in space comprises any of the following steps or combinations thereof:
A. Binding the biological molecules to a known or unknown immobilized array of molecules.
B. Separating the biomolecules with one- or two-dimensional electrophoresis (gels).
C. Attaching the biomolecules to a membrane (blot).
D. Observing the biomolecules' affiliation to a specific cell.
E. Separating the biomolecules by chromatography.
F. Separating the biomolecules in a flow system.

The step of tagging the biomolecules with markers can be done before or after the above described stages, depending on the case. When the biomolecules are DNA molecules, the tagging is preferably done before the spatial separation, while for proteins in the 2D-gel or microarrays the tagging is preferably done after the spatial separation.

The tagging may comprises any combination of the following alternatives:
A. Use of heavy metal colloids (e.g., gold, silver), preferably of a diameter range of 1-200 nm, whereby the colloids create a high intensity back scattered electron signal and, therefore, high image contrast.
B. Doping the biological molecule with a high atomic number substance, to create a contrast sufficiently strong under an electron beam. Preferably the doping substance is an organic compound that contains iron.
C. A fluorescent electro-luminescent signal whereby the electron beam creates a light signal that is detected. Such marking has the advantage of high resolution electron beam inspection with optical reading.

Tagging with more than one type of tags to make a distinction between two preparates or different molecules is also possible. In the case of colloids, the tags may comprise different materials or of different sizes. The material analysis can be done with EDS (Energy Dispersive Spectroscopy) yielding an X-ray spectrum. The differentiation on the basis of size is done by image analysis.

For preparing the preparate for vacuum standard methods are used that include:
A. Fixation e.g., with formaldehyde.
B. Coating with a conductive layer e.g., carbon.
C. Freezing to prevent out-gassing.

The preparation of the preparate for vacuum can be minimal when using an Environmental Scanning Electron Microscope (ESEM), as described further below.

After loading the preparate in the specimen chamber of an electron microscope, scanning of the preparate is carried out by reading at low resolution either by the electron beam or with a combined optical microscope, including optical fiber where access is indirect. In this way, the regions of interest (ROIs) are identified and are then scanned at a higher or highest resolution.

The scanning of the ROIs with an electron beam at high resolution can be in one or two dimensions. The scanning in one dimension can be synchronous with the movement of the preparate in the second horizontal dimension.

To enhance the image contrast, algorithms well-known in the art can be used such as, for example, by calculating the histogram of pixel values and expanding it over the dynamic range of pixel values (typically 2-255).

The analysis of the ROIs comprises any one of the following steps:
A. Performing edge detection algorithm to identify the colloids in each ROI and counting said colloids.
B. Counting electro-luminescent signals.
C. Identifying X-ray spectrum of each particle for identification by comparison to a reference spectrum.
D. Counting the signals by image analysis.

Display of the results may include presenting the number of molecules in each ROI and the spatial map of the ROIs with indication of the different types of molecules.

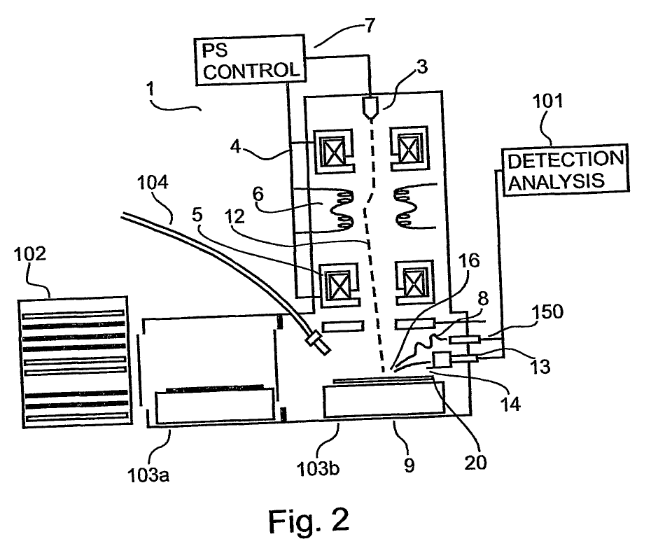

Reference is now made to FIG. 2 which is a general diagram showing a longitudinal cross-section of a SEM 1 used as a preparate analysis apparatus according to the present invention. Instruments similar to SEM 1 are used for inspection of semiconductor wafers, as described, for example, in U.S. Pat. Nos. 6,072,178, 5,644,132, 5,502,306, 4,618,938, 4,609,809 and 4,618,938, the contents of which are herein incorporated by reference as if fully disclosed herein.

A primary electron beam 12, travels through a vacuum path to reach a preparate 20. The electrons are emitted from an electron gun 3, powered by a gun power supply which is electronically controlled as indicated by 7. The beam is focused by a condenser lens 4 and an objective lens 5, to form a focal point on preparate 20. The beam is diverted by a deflector 6 that scans the preparate in one or two dimensions. The preparate emits secondary electrons (SE) 14, back scattered electrons (BSE) 16 and characteristic X-rays 8. The characteristic X-rays are subjected to energy analysis to form and X-ray spectrum via an EDS. The BSE are detected by the BSE detector 18. SE 14 are detected by the SE detector 13. X-rays are detected by the EDS detector 150. The analog data is acquired and analyzed in a computer schematically represented by box 101. Optical microscope assisted with optical fibers 104 is used for low resolution inspection. A preparate that comprises biological molecules is fed via a tray 102 into the SEM. The preparate compartment 103 is divided to two sections in each of which pressurization can be performed independently to permit loading or unloading of a preparate in one chamber (e.g., 103A) while simultaneously inspecting a second preparate (in e.g., 103B). Each of chambers 103A and 103B are large enough to contain a 12″ (30 cm) diameter preparate 8. A stage 9 drives the preparate under the scanning electron beam 12. The image is formed from the detected electron signal and is digitized for image processing.

Preparate 20 contains biological molecules that are separated in space. According to one embodiment of the present invention, preparate 20 comprises a hybridized DNA microarray, as detailed further below. According to another embodiment of the present invention, the preparate comprises a 2D PAGE as also detailed below. According to still another embodiment of the present invention, the preparate comprises a protein microarray, a carbohydrate microarray or a cell microarray interacted with any probe or target.

Reference is now made to FIG. 3, a generalized diagram showing a cross-section of a scanning electron microscope according to one aspect of the present invention. Parts that are the same as those shown in the previous Figure are given the same reference numerals and are not described again, except as necessary for an understanding of the present embodiment.

According to this aspect of the present invention preparate 20 comprises a DNA microarray 70, after hybridization. The substrate 72 is standard, e.g., glass, nitrocellulose membrane, nylon filter, filter paper, other substrates that are used in Southern, western or northern blots or in microarrays. The substrate can be silicon, glass, filter paper or any other material that is convenient for microarray fabrication. The miss-matched and perfect-match target-probes are marked by 26A and 26B, respectively. The technique of gold tags (discussed further below) is used.

In order to prevent charge accumulation, the microarray is coated by a thin layer of a conductive material, schematically shown by the dashed line of 73. Preferably the coating material is carbon. Preferably the thickness range is 50 to 600 A. In order to make the preparate vacuum compatible, it is fixed by standard electron microscopy methods, preferably by using formaldehyde. An alternative method to increase the compatibility between the microarray 70 and the vacuum is by using a cooling stage 74, preferably based on the Peltier effect. The stage is used to cool the microarray and thereby reduce the out-gassing.

The specific microarray preparate comprises domains of different probes 26 as shown in FIG. 3 by way of a non-limiting example. A domain of base structure TTGC 26A (SEQ ID NO:1), is shown as a representation of a miss-match. A domain of ATGC 26B (SEQ ID NO:2) represents perfectly matched probes. The probes are hybridized with the target molecules 27. A plurality of tags 28 (e.g., gold colloids) is attached to the target molecules. In a preferred embodiment, there is one tag per target. The advantage of gold is that it creates a strong signal of back-scattered electrons due to its high atomic weight and corresponding high back-scattered electrons coefficient. This allows a high contrast, high resolution image at low current and exposure time, thus minimizing the radiation damage to the preparate.

It is possible to obtain commercial gold colloids as small as I nm (for example, from Nanoprobes, Inc., 95 Horse Block Road, Yaphank, N.Y. 11980-9710, USA). In order to get good correlation between the number of gold particles and the number of hybridized DNA, it is important that the number of colloids per target will be constant, preferably one tag per probe. The binding between tags and targets should be stable at a temperature of 50° C., which is typical for hybridization. According to a yet another embodiment of the present invention, the tags are iron-rich molecules, as in the technology used by Clinical Micro Sensors, Inc. (126 West Del Mar Blvd, Pasadena, Calif. 91105, USA). The iron produces a sufficient contrast for the BSE signal.

The SEM/gold tags combination along with the ability to drive the preparate mechanically enables quantitative microarray detection. According to the present invention, the colloids are counted from the acquired digital image. Then, the contrast of the image is enhanced using a contrast enhancement algorithm. Then a pattern recognition algorithm identifies the colloids, for example by edge detection. Subsequently, the colloids are counted. This provides a quantitative measure of even the weakly expressed genes. A preferred reading strategy that reduces the reading time is to first scan the entire preparate and identify the regions of interest and then rescan the regions of interest to the desired quality. The ability of SEM to work at resolutions that vary from 10 microns down to about 1 nm, provides an ability to perform single molecule detection on a very wide dynamic range.

The present invention is advantageous over existing methods since it is based on single-molecule detection. This means that the sensitivity and dynamic range are considerably higher than in the presently used fluorescence based methods. One advantage of single molecule detection scheme is the fact that it is compatible with miniaturization of a microarray. The miniaturization is desired since one would like to pack compactly as many molecules as possible on the same chip.

The following table summarizes a comparison of performance of the single molecule detection method of the present invention and the alternative fluorescence technology. A further advantage that relates to the miniaturization is the ability to use smaller preparates. In many cases only a limited amount of sample is available. Exponential amplification methods such as PCR may alter the results in an uncontrolled manner. The advantages of a sensitive system that can detect smaller preparates are clear.

TABLE 1

| | Fluorescence | Electron Beam |
|---|---|---|
| Detection Comparison: | Detection | Detection |
| Resolution (pixel size) [unit] | ˜10 μm | <10 nm to ˜10 μm |
| Scan Time [min] | ˜18 | ˜ comparable (depends |
| | | on number and size of |
| | | ROIs) 40 min to scan a |
| | | whole microarray at a 100 |
| | | nm resolution |
| Sensitivity | ˜10⁵ | 1 |
| [molecules/100 μm²] | | |
| (Number of molecules per | | |
| micron squere required for a | | |
| signal) | | |
| S/N | ˜3-4 | Much better, e.g., 20-100 |
| Dynamic range of linearity | ˜10³ | ˜10⁸ |
| Can rescan preparates | N | Y |
| (no bleaching) | | |
| Compatible with small | N | Y |
| preparates (reducing PCR | | |
| steps) | | |
| Compatible with miniaturized | N | Y |
| arrays | | |

In another embodiment, the method of the present invention can be carried out at almost atmospheric pressure, for example using an Environmental Scanning Electron Microscope (ESEM), a commercial SEM that works at elevated pressures. Further information on ESEM and how it works can be found in Enviromnental Scanning Electron Microscopy, Philips Electron Optics, Eindhoven, The Netherlands (Robert Johnson Assoc. El Dorado Hills, Calif. 1996) as well as in U.S. Pat. Nos. 5,250,808, 5,362,964 and 5,412,21 1, the contents of which are hereby incorporated by reference as if filly disclosed herein.

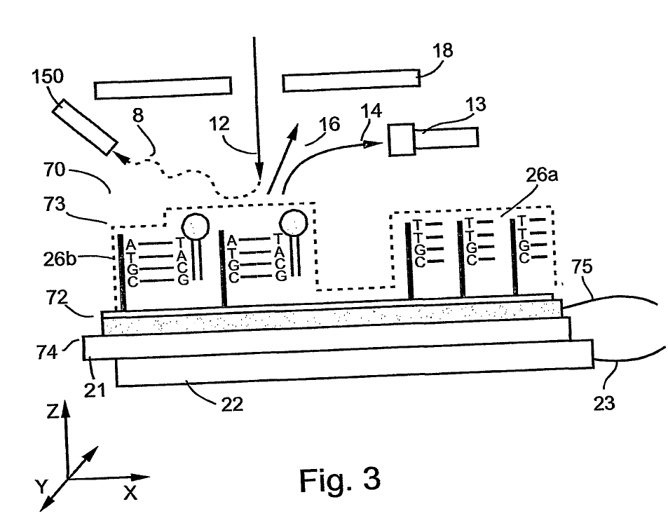

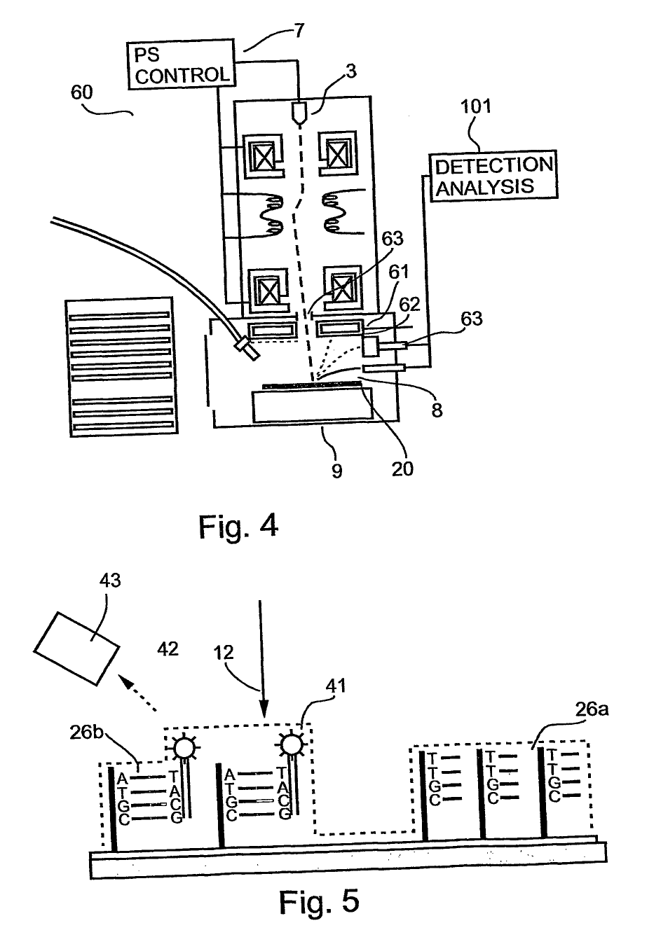

According to a yet another embodiment of the present invention, the ESEM is used for the inspection of the microarray of FIG. 2., without the preparation for vacuum. In a preferred embodiment, the microarray will be cooled by cooling plate 74, to reduce the vapor pressure. The main advantage of the ESEM is the ability to study topography in an elevated pressure, utilizing a prior art secondary electrons (SE) detector. The advantage of the ESEM is that the hybridization can be detected simply by measuring the density at each site. Thus, as mentioned below, according to one embodiment, the microarray will be analyzed without tagging the targets. According to another embodiment of the present invention, the ESEM, or its specimen compartment, will be used instead of the SEM disclosed in FIG. 2. The lower vacuum and the cooling relaxes the steps needed to prepare the preparate for vacuum. For example, when the ESEM is used, it is possible to inspect 2D PAGEs and microarrays without fixation. An example of an apparatus that combined the ESEM with the SEM disclosed above is given in FIG. 4. The SEM 60 contains a pressure limiting aperture 63 that distinguishes between specimen compartment 103 and the column. In order to protect the back scattered electrons detector, it is enclosed in a protective chamber 61, the front window of chamber 62, is a membrane transparent to electrons that can hold the pressure difference between the evacuated medium near the detector and the gas. The secondary electrons are detected by the prior art ESEM SE detector.

According to a yet another embodiment of the present invention the targets are not tagged at-all. The SEM is sufficiently sensitive to detect density differences between hybridized and non-hybridized regions in nucleic acid microarrays and, similarly, interacted vs. non-interacted molecules in other types of microarrays, including protein and carbohydrate microarrays. A difficulty in using an electron beam 12 for biomolecules is that it may damage the preparate. The damage to DNA, for example, from a beam of electrons is described in “Measurement of DNA damage by electrons with energies between 25 and 4000 eV”, Folkard et al., Int J. Radiat. Biol. 64(6) pp 651-658 (1993). The choice of parameters should be safely below the damage. The use of gold allows one to use small probe currents, in the range of 10 pA, and fast scanning, typically 10 or more frames per second, thus minimizing the radiation absorbed by the DNA. In the areas where gold is present, it is expected that most of the interaction will be with the heavy gold atoms and not with the biomolecules. The main danger of radiation is the formation of free radicals in the water. Hence, according to a preferred embodiment of the present invention, a chemical that reduces the formation of free radicals but does not damage the biomolecules is added to the microarray after hybridization/interaction and before inspection. Further information on these chemicals can be found in Siddiqi M. A. and Bothe E., Radiation Research, Vol. 112, pp 449-463 (1987), the contents of which are herein incorporated by reference.

Preferably, the microarray preparate is situated on a stage 21 that can be moved by a servo motor 22. This arrangement drives the preparate under the electron beam and effectively increases the scanned area. The motor may incorporate an electrical vacuum feed-through 23. Such a motor is commercially available from Nanomotion, Ltd. (Mordot HaCarmel Industrial Park, PO BOX 223, Yokneam, 20692, Israel). Alternatively, the stage can be moved by a conventional mechanical feed through. According to yet another embodiment of the present invention motor 22 is situated on an arm that drives it into the vacuum chamber via a load lock. Such an arrangement improves the automation and elevates the throughput of the system. According to a preferred embodiment of the present invention, the preparate is driven along one axis (marked by X) and the electrons beam scans the microarray along the perpendicular axis (marked by Y).

Due to the low signal-to-noise ratio inherent to microarrays, protein microarrays in particular, as well as to other methods currently used to detect biological species, it is desirable to use comparative, rather than absolute measures. In the commonly used fluorescence-based microarrays, this is done on the basis of different dye colors. According to a yet another embodiment of the present invention, this is done in the SEM by means of an X-ray spectrum analysis. The X-ray is detected by an EDS detector. The X-ray photons and the detector are marked in FIG. 2 by 8 and 150, respectively. The characteristic X-ray spectrum of the tags serves for comparative study. As the colloids are counted, their spectrum is acquired, analyzed and compared to a reference spectrum. This method allows comparative study of more than one tag since the number of possible X-ray spectrums is not limited.

Reference is now made to FIG. 5, that shows a yet another embodiment of the present invention, whereby the detection is made by exciting light photons (electro-luminescence). The target molecules are tagged with luminescent molecules 41, in a similar fashion to the prior art fluorescent tagging. According to the present invention, the luminescent tags 41 are excited by the electron beam 12 and/or the excited SE. The light beam 42 is guided to a photomultiplier (PMT), by means of a light guide 43 (e.g., made of PMMA). The amplified light signal produced by the PMT is transformed to an electrical signal at the SEM detector. The device allows inspection of light emitted from the fluorescent DNA molecules, at a resolution below 100 nm.

A further development that will increase throughput is parallel inspection with microcolumns. The microcolumns are miniature scanning electron microscopes that are produced by integrated silicon processes. Due to their size, the microcolumns can operate in parallel, considerably reducing the scanning time and the bulkiness of a SEM based system. Further details of the microcolumns are given in A. D. Feinerman and Crewe “Miniature Electron Optics”, Advances in Imaging and Electron Physics, Vol. 102, 187 (1998) as well as U.S. Pat. No. 5,122,663, the contents of which are hereby incorporated by reference as if fully disclosed herein.

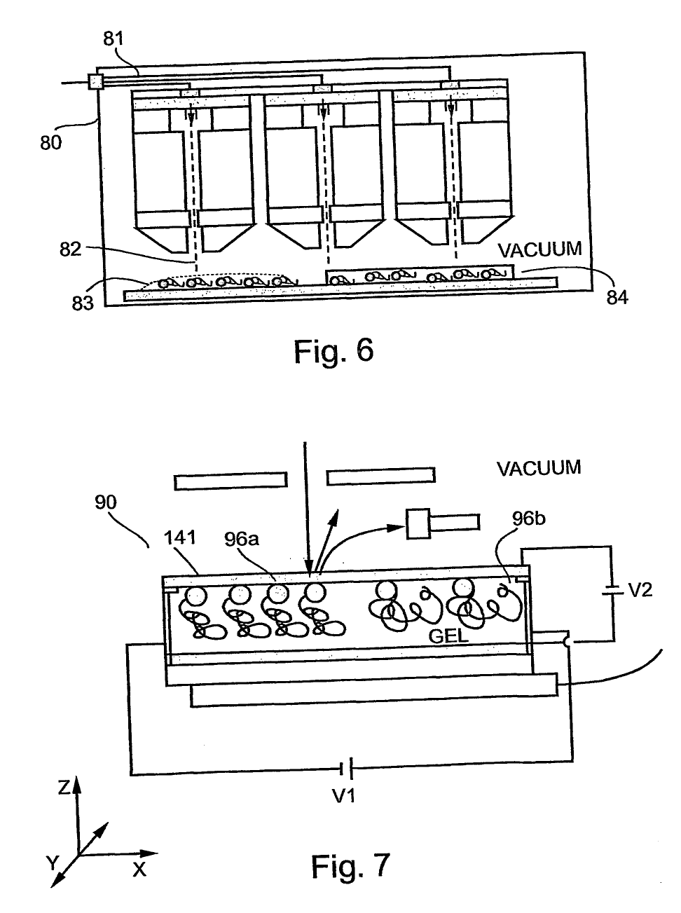

Reference is now made to FIG. 6. According to the present invention, a plurality of microcolumns arranged in an array 80 is used for analysis of genes or proteins. Electrical wiring 81 controls the electron beam and lead the information from the detector to the data processing system. The beam of electrons scans the preparate of tagged nucleic acid, proteins, carbohydrates or cells in a microarray and/or 2D-gel, as appropriate. According to one embodiment of the present invention, the biological preparate can be coated with a conducting material e.g., carbon, as shown in 83. Alternatively, the preparate may be protected in a close chamber, as shown at 84 and the electrons will travel through the membrane.

According to an embodiment of the present invention, the SEM is used for the analysis of proteins in a 2D PAGE. According to this embodiment, after counting, the preparate is prepared for mass spectrometry. According to this embodiment, the separation of proteins is done in a 2D PAGE. However, the present invention is compatible with other separation methods, for example, electrophoresis in a fluid or through a membrane (e.g., as in a blot), chromatography (HPLC). The typical size of a 2D PAGE is 20×20 cm². This means that the entire 2D PAGE can easily fit into the standard wafer compartment of a wafer inspection SEM (103 in FIG. 2.).

According to the present invention, the preferred tagging method is the attachment of gold colloids. This is done with known technologies such as, for example, the one available from British Bio Cell Inc. (Cambridge, GB).

According to another aspect of the present invention tagging is done by silver staining. Since there is no generic tagging that fits all proteins, the type of tagging to be used depends on the biological question that is asked. In many cases, the relevant question is whether a known protein exists in a preparate. In this case, the specific tagging of this protein, or number of proteins is applied and the desired proteins can be read on a single molecule detection basis. In other cases, general tagging, such as or silver staining can be applied.

According to the present invention, the preparation for vacuum is done as follows: first the tagged molecules (proteins) are driven to the surface by an electric field (shown schematically in FIG. 7 which is further referred to hereinbelow) and then the proteins are immobilized on the surface. According to one embodiment of the present invention, the surface is made of silicon. According to another embodiment, the surface is made of glass. After the proteins are attached to the surface, the surface is detached from the gel, coated to prevent charge accumulation by the electron beam of the electron microscope and then scanned thereby.

According to the present invention, the scanning is done by driving the preparate mechanically under the electron beam in a continuous or a ‘step and repeat’ manner. The driving is done in correlation with the—scanning. According to one aspect of the present invention, the scanning is first done at a low resolution, to identify the ROIs, either automatically via the software or manually. Then the preparate is scanned at a higher resolution to count the number of colloids in the significant spots.

Reference is now made to FIG. 7. The gel chamber 90 comprises 3 sets of electrodes. The electric field that separates the molecules is applied by power supply V1 in the X direction and V3 in the Y direction. The attachment to the upper surface is done via V2 (Z direction).

According to another embodiment of the present invention, the proteins are marked with fluorescent or electro-luminescent molecules, similar to the embodiment disclosed in FIG. 5 for DNA microarrays. According to one embodiment of the present invention, the molecules are tagged before they are separated in the gel or attached to the microarray. Typically protein analysis consists of two typical phases: separation or localization in space and identification via mass spectrometry, antibodies, etc. What is clearly missing is an intermediate stage where the number of proteins in each spot is counted. Preferably the counting method will be able to distinguish between different types of proteins.

According to the present invention there is disclosed a method of protein analysis that comprises of the following phases:
1. Localization via a 2D PAGE or on a microarray
2. Quantification (in an SEM)
3. Identification (preferably via mass spectrometry, specifically Matrix Assisted Laser Dissociation/Ionization-MALDI, antibodies).

In another embodiment of the present invention, proteins or protein samples, which can include proteins of a known identity or of an unknown identity, naturally occurring or synthetic, antigens or antibodies, etc., are arrayed over a surface of a microarray and are immobilized thereto and are thereafter interacted with appropriate directly or indirectly tagged macromolecules to generate a preparate suitable for electron microscope inspection. Other preparate processing steps are similar to the steps described elsewhere herein.

In yet another embodiment of the present invention, saccharides or saccharide samples, which can include saccharides of a known identity or of an unknown identity, naturally occurring or synthetic, are arrayed over a surface of a microarray and are immobilized thereto and are thereafter interacted with appropriate directly or indirectly tagged macromolecules to generate a preparate suitable for electron microscope inspection. Other preparate processing steps are similar to the steps described elsewhere herein.

In still another embodiment of the present invention, cells of a known identity or of an unknown identity are arrayed over a surface of a microarray and are immobilized thereto and are thereafter interacted with appropriate directly or indirectly tagged macromolecules to generate a preparate suitable for electron microscope inspection. Other preparate processing steps are similar to the steps described elsewhere herein.

According to the present invention, the disclosed apparatus and method can be used for building databases. For example a database that aim at interfacing protein information with DNA mapping and sequence data from genome projects. This may also include a file listing all of the information entered for the particular protein. An example of such a database, obtained by conventional means is described in: J. E. Celis, FEBS Letters 430, 64-72, 1998 which is incorporated herein by reference.

According to a preferred embodiment of the present invention a wafer-inspection SEM is used to detect labeled molecules on microarrays.

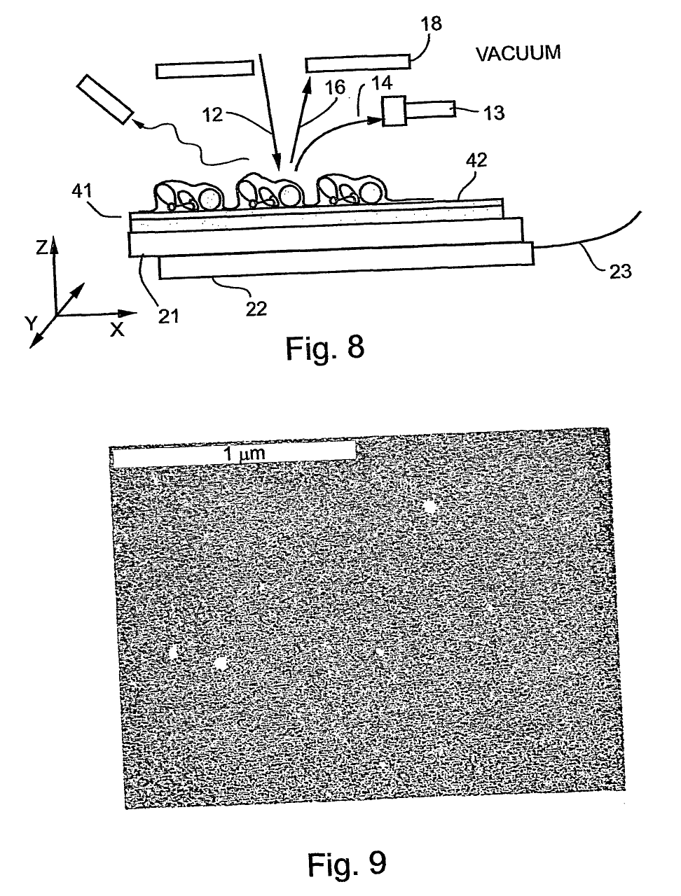

Reference is now made to FIG. 8. The tagged proteins are immobilized on a surface 141. According to a preferred embodiment, the surface 141 is coated with a thin layer of carbon 142. After immobilizing the proteins, the substrate is coated with an additional layer of carbon to prevent charge accumulation in the proteins. The substrate is then scanned in the SEM.

Reference is now made to FIG. 9, showing gold conjugate proteins as imaged in the SEM. The image is shown ‘bare’ without contrast enhancement. It can be seen that an image analysis technique can be applied to quantify the number of tags. This experiment has been performed with monoclonal antibody (mouse IgG1) 1E10 conjugated to 20 nm gold colloids. A silicon substrate was covered with a carbon layer of 150-200 Angstrom. The substrate was attached to a conventional SEM aluminum support. On the silicon surface was a drop of antigen P277. The drop was dried in a vacuum oven at 40° C. for 20 minutes. The gold conjugated antibody was added by putting a drop on the silicon surface, in a way that covered all the surface. The antibody added was diluted 1:10. The support was left in a humid chamber for 40 minutes. The antibody was washed by dipping the support for a few seconds in PBS (phosphate-buffered saline) a few times and then in double-distilled water.

As is discussed hereinabove and is further exemplified in the Examples section that follows, the present invention teaches a method that reaches single molecule detection levels, gives high signal-to-noise ratios, and demonstrates a very broad dynamic range, while retaining easy preparate preparation. In its preferred embodiment, the method uses gold labeling and electron microscopy, preferably a Wafer-Inspection Scanning Electron Microscope, to probe both protein function arrays and protein detecting arrays and is demonstrated here using the same immobilization chemistry and robotics described by the prior art (G. MacBeath, Science 298 (2000) 1760-1763).

The reasons gold labeling and SEM scanning demonstrate such abilities are several and include:
1. Significant reduction of noise. Unlike fluorescence techniques where auto fluorescence of the glass and the buffers contribute to the noise, in the method of the present invention false positive signals are only due to nonspecific binding of the gold conjugated probe to the slide. This is because only signal (back scattered electrons) coming from very heavy atoms such as gold are detected by the SEM detectors.
2. In small enough dilutions, where the molecules attached to the slide are in distances larger than the diameter of the gold probe, only one gold particle can attach per probed protein or ligand. This is because once a gold particle attached, it occludes the molecule on the surface from other gold probes. Since the SEM can detect single gold particles, this provides single molecule detection capabilities.
3. Detection abilities are limited in the lower limit only by false positive signals due to non-specific binding, and in the upper limit by the highest number of colloids able to pack closely in a given area in other words, the upper detection limit can be controlled by the size of the gold colloids chosen as probes. This ensures a very broad and highly linear dynamic range. Also, detection abilities are not constrained by instrumentation (unlike fluorescent methods where saturation of the photodiodes in light detectors can occur).

In addition to the increased abilities in sensitivity and dynamic range there are several other advantages over other labeling methods such as fluorescence:
1. There is no bleaching of the signal. This means that the preparate can be stored and scanned repeatedly across a long period of time (e.g., in cases that inconsistencies were found in the analysis or new data has accumulated that requires new analysis of an old preparate).
2. The preparates have a higher reproducibility rate since there is no dependence on the type of buffers and materials used, and there is a weak dependence on the “Hands” preparing the preparate.
3. In the case of gold tagged proteins, it seems the gold enhances binding selectivity of protein bound to it, possibly because in some cases there are several proteins per gold which can increase the probability of binding. Another possibility is that due to the large size of the gold only high affinity and specific interactions survive the stringent washing conditions.

The potential of the method of the present invention for relatively high throughput is demonstrated in the unrelated semiconductor industry where SEMs are routinely used to scan wafers for microscopic defects and impurities.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which describe experiments aimed at testing the sensitivity (signal-to-noise ratio) and the dynamic range of the method of the present invention. To this end, first the universal biotin-tagged avidin detection system was tested comparatively to the prior art, to reveal that under various experimental conditions sensitivity is up to 100 fold improved, whereas the dynamic range is both several orders-of-magnitude broader, with a lower limit reaching the ultimate goal of single molecule detection. Then, the universal biotin-tagged avidin detection system was sandwiched to a hapten-antibody detection system and tested for its sensitivity and dynamic range.

### Materials and Experimental Procedures

Arraying Proteins on Glass Slides:

Proteins were spotted on glass slides presenting aldehyde groups (Telechem International, SuperAldehyde Substrates) using a Biorobotics TAS arrayer. Three “flag” proteins (Biotin-BSA at a concentration of 0.1 mg/ml) served as position pointers for each group of 4×4 spots. Spotted drops were about 400 μm in diameter and 10 nano-liter (nl) in volume. 256 protein spots were applied to each spotted area of a slide. For fixation via the aldehyde groups, the spotted slides were incubated in a humid chamber for 2-3 hours at ambient temperature. When required, spotted areas and/or subareas of a slide were spatially separated by surrounding paraffin lines. In order to block free aldehyde groups, the slides were inverted and briefly placed in a solution of 1% BSA in PBS, pH 7.5, and then immediately immersed in a similar fresh solution for 1 hour at room temperature with gentle agitation. Following a brief rinse in PBS, the slides were ready for further processing as described below.

Probing the Slides:

Gold conjugated streptavidin (gold-streptavidin) was purchased from British Biocell International. Gold colloids were 20 nm (STP20) or 40 nm (STP40) in diameter. Gold-streptavidin was spun four times in a cooled centrifuge at 12,000 revolutions-per-minute (RPM) for 20 minutes, resuspended twice in a fresh buffer containing 0.04% Tween20 (Sigma) and 0.1% BSA (w/v); and twice in a fresh buffer containing 20% glycerol, 80% PBS, 0.1% BSA (w/v) and 0.5 M NaCl. Final concentration of gold-streptavidin was approximately 15 nM for both STP20 and STP40.

Cy3-conjugated streptavidin was purchased from Amersham Pharmacia Biotech and diluted in a solution of 20% glycerol, 80% PBS, 0.1% BSA (w/v) to a concentration of 17 nM.

Slides were incubated for 4 hours with the gold-streptavidin, or Cy3-streptavidin probes.

Preparing Gold Probed Slides for Electron Microscopy:

All gold-probed slides were immersed in a fixation/dehydration solution (3% paraformaldehyde and 2% glutaraldehyde) for 30 minutes, washed in double distilled water (DDW) for an additional 10 minutes, spun for 5 minutes at 1250 RPM to remove excess buffer, and dried in a vacuum chamber overnight. The slides were then cut into 1 inch² sections that contained all the spotted area, using a diamond glasscutter and attached to electron microscope supports via a carbon double-sided tape. In order to avoid charging of the samples, the glass sections were coated with a 200 nm thin carbon coat using an Edward's carbon coater and their edges were colored with a conducting silver paste.

Scanning the Probed Slides:

Fluorescence of the Cy3-probed slides was scanned using a Packard ScanArray 4000 scanner at a 20 μm resolution. Intensity was determined by taking the average intensity of the pixels in corresponding spots in all slides and reducing the average intensity of the pixels immediately surrounding the corresponding spots. Typically, each data point was based on four individual experiments.

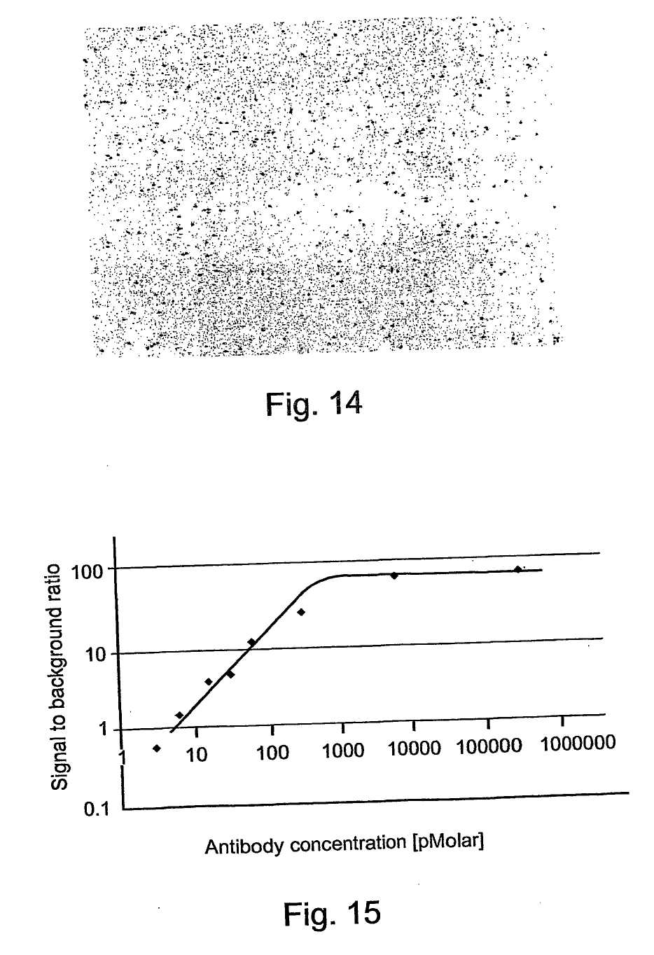

The gold-probed slides were visualized with a scanning electron microscope (SEM) (Jeol 6400).. Images of 48 μm² (for 40 nm gold colloids) or 12 μm² (for 20 nm gold colloids, see FIG. 14) sized frames inside each spot were taken via a back scattered electrons detector. This detector can detect only electrons scattered from heavy atoms, and therefore detects only the gold colloids and not any organic, light weight atoms present. Gold colloids were counted using the NIH image processing software (shareware downloadable from: http://www.pathsoc.org.uk/wwwboard/messages/214.html). Number of gold colloids per spot, were taken to be the average number of gold colloids in corresponding frames multiplied by the number of frames per spot (2.6103 of 48 μm² sized frames per spot in the case of 40 nm gold colloids; 10.4-10³ of 12 μm² sized frames per spot in the case of 20 nm gold colloids).

BSA-Biotin—Gold/Cy3-Streptavidin Detection System:

BSA and biotin-caproate were purchased from Sigma. BSA-biotin conjugate was prepared as follows: BSA (5 mg, 75 nmole) and biotin-caproate, (0.72 mg, 1.9 μmole) were dissolved in ice cold 200 μl DMF and the mixture was left at room temperature for 2 hours. The number of biotin molecules per BSA molecule, estimating 50% conjugation efficacy is 12.5 on the average. Extensive dialysis was preformed against PBS to remove unconjugated biotin. Activity of the BSA-biotin was assayed employing ELISA, using Horse Radish Peroxide conjugated to streptavidin as a probe.

BSA-biotin, and BSA were dissolved in 40% glycerol, 60% PBS to an initial concentration of 1 mg/ml. BSA-biotin was serially diluted 3-fold in 40% glycerol, 60% PBS, pH 7.5, 0.1% BSA (w/v). In all dilutions, the total amount of BSA (free BSA+biotinylated BSA) was kept constant at 1 mg/ml. BSA-biotin was then spotted on the slides in different concentrations ranging from 1 mg/ml to 100 ng/ml. Free BSA, which served as a control was also spotted on the slides. Slides were than processed as described above.

To probe the slides, 40 μl of gold-streptavidin (STP20 or STP40) or Cy3-streptavidin were applied to each printed area and incubated for 4 hours in a humid chamber at ambient temperature. Following incubation, the slides were washed 3 times, 3 minutes each time, with PBS supplemented with 0.04% Tween20 (PBS/T). Cy3-streptavidin probed slides were additionally rinsed twice in PBS, (3 minutes each rinse), centrifuged for 5 minutes at 1250 RPM to remove excess buffer, and left to dry in a slide box. Gold-streptavidin probed slides were further processed for electron microscopy as described above. Slides were scanned as described above.

BSA-Hapten—Biotinylated Antibody—Gold Streptavidin Sandwiched Detection System:

BSA-hapten 23.7 [1b (p-nitrobenzyl phosphonate N-glycylglutatarate)] was prepared as described in Tawfik et al. Phosphorus and Sulfur, 1993, vol. 76 123-126.

D2.3 antibody was prepared as described in Tawfik et al. Proc. Natl. Acad. Sci. USA, 1993, vol. 90 p. 373-377.

The affinity constant for hapten 23.7 and D2.3 antibody in solution was determined to be 4 nM by competitive ELISA (Tawfik et al. (1997) Eur. J. Biochem. vol. 244 p. 619-626) and further by a fluorescence assay (Lindner et al. (1999) J. Mol. Biol. vol. 285 p. 421-430).

Biotin-caproate was purchased from Sigma. 90 μl of biotin-caproate (20 μmole) were dissolved in a solution having a total volume of 1 ml and containing 336 μl D2.3 antibody (1 mg, 6.7 nmole) in PBS and NaHCO₃ (1 M; 100 μl). The reaction mixture was placed on ice for 3 hours, followed by extensive dialysis against PBS. Activity of the D2.3 antibody was assayed with ELISA SA-HRP/GaM HRP.

BSA-hapten 23.7 was dissolved in 40% glycerol, 60% PBS, pH 7.5 at a concentration of 200 μg/ml and spotted on slides as described above. The slides were then further processed as described above.

The control, non biotinylated D2.3 (5 ng/ml), was dissolved in 20% glycerol, 80% PBS, 0.1% BSA (w/v). The biotinylated D2.3 antibody was diluted in a solution containing 20% glycerol, 80% PBS and 0.1% BSA (w/v). Dilutions ranged from 50 μg/ml biotinylated D2.3 antibody to 0.5 ng/ml biotinylated D2.3 antibody. Twenty μl of each dilution and control were applied to separate sections of the slides. Following 2-3 hour incubation in a humid chamber at ambient temperature, the slides were washed 3 timed (3 minutes each wash) with PBS/T.

To probe the slides, 40 μl of gold-streptavidin were applied to each printed area and incubated for 4 hours in a humid chamber at ambient temperature. Following incubation, the slides were washed 3 times for 3 minutes each time in PBS/T. Gold-streptavidin probed slides were further processed for electron microscopy as described above. All slides were scanned as described above.

### Experimental Results

BSA-Biotin—Gold/Cy3-Streplavidin Universal Detection Systems:

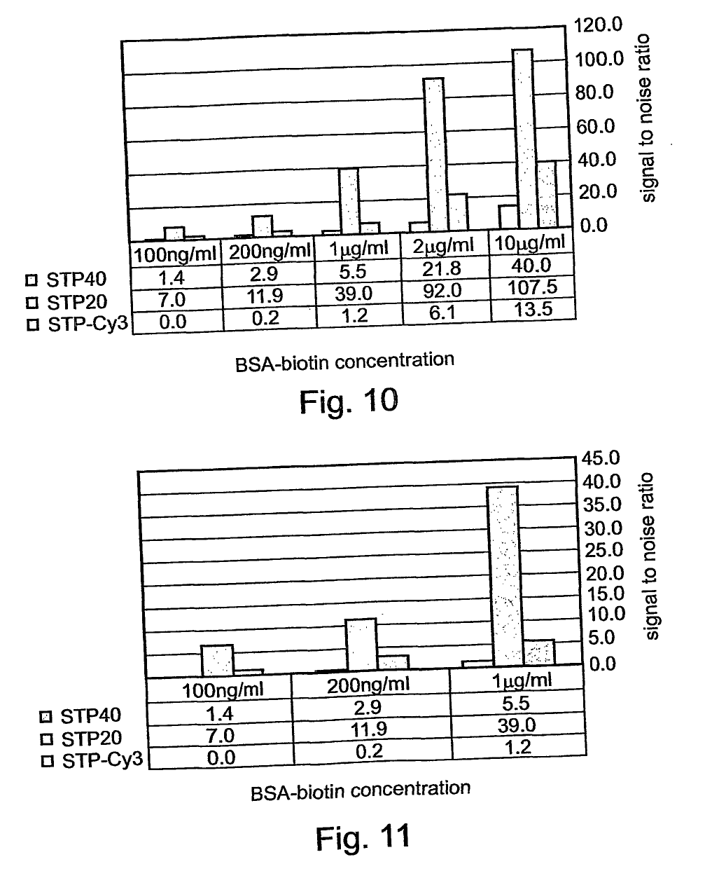

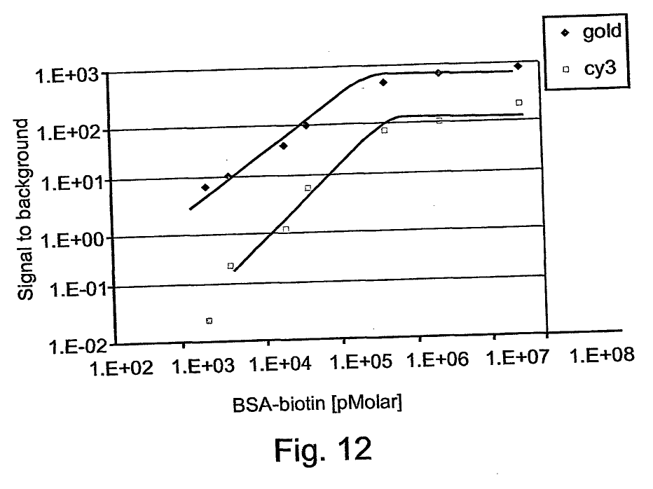

The results for the BSA-biotin—gold/Cy3-streptavidin universal detection system are shown in FIGS. 10-14, clearly demonstrating the far superior sensitivity (higher S/N ratios) and dynamic range of the present invention over the prior art in any and all of the experimental conditions employed. A few interesting points arise from the results. The first is that gold probes (STP20) extend the dynamic range (i.e., the range where signal scales linearly with protein concentration) and the sensitivity of detection by almost 100-fold relative to fluorescence probes (see, FIGS. 10-12). Another result is that 20 nm gold colloids performed far better than 40 nm gold colloids (improving signal-to-noise by about 4-fold, or [R40/R20]2, see FIG. 10 and 11). The latter result indicates that using even smaller gold colloids, e.g., 10 nm and 5 nm colloids, will improve sensitivity and extend the lower limit of the dynamic range by an additional 10-100 fold relative to the fluorescence probe. Since the largest number of colloids able to pack closely in a given area limits the upper detection limit, it is expected that decreasing the size of the colloids from 20 nm to 5 nm will further extend the dynamic range in its upper limit by a factor of at least 16 fold. Moreover, by careful normalization of measured results, it will be possible to carry out relative measurements of protein samples on the same microarray using distinguishable sizes of colloids.

In order to approximate the detection abilities of the system, the ratio between the approximated number of BSA-biotin molecules conjugated to the glass surface in one spot, and the number of gold colloids detected in a spot was evaluated. To estimate the number of biotin labeled BSA molecules conjugated to the glass in each spot, first the number of aldehyde groups per spot was calculated by multiplying the number of aldehyde groups per cm² (5·10¹² groups per cm²) by the area covered by a drop of a 400 μm in diameter (1.25·10⁻³ cm²). The result is 6·10⁹ aldehyde groups per spot. The number of BSA molecules contained in a 10 nl droplet of a 1 mg/ml BSA solution is 9·10¹⁰ molecules, which means that a maximal attachment of <10% of the protein molecules in a droplet can be achieved. To estimate the number of gold colloids detected in a spot, the average number of gold colloids counted per frame was multiplied by the number of frames contained in the area covered by the drop (see above).

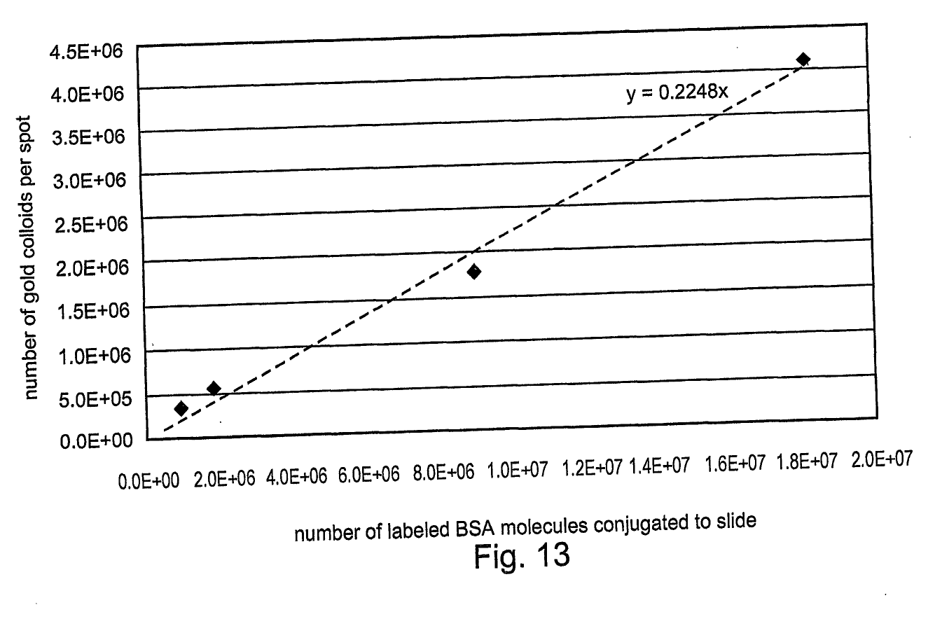

FIG. 13 presents the estimated numbers, assuming a maximal attachment of 10%. Taking the slope of the linear fit, and taking into consideration that the aldehyde quantification was not done by protein attachment but rather by attachment of small molecules, the real value of protein attachment is probably <10%, hence, the detection is close to 1:1 of all molecules present in a spot. In other words, assuming that 10% of the biotin molecules floating in the spotted drop also conjugate successfully to the glass surface via the aldehyde groups thereat, the detection is at worst 1 of every 4, but more likely closer to detecting all biotin molecules. This experiment demonstrates that by using the method of the present invention, the lower possible limit of the dynamic range, i.e., every single molecule detection, was reached or nearly reached.

BSA-Hapten—Biolinylated Antibody—Gold Streptavidin Sandwiched Detection System:

To determine the sensitivity to concentration and as a demonstrative application for the method of the present invention a model system based on a sandwich detection system—BSA-hapten—biotinylated antibody—gold/Cy3 streptavidin—was employed. In the model system, a hapten conjugated to BSA was spotted on glass slides. It was then interacted with different concentrations of a corresponding biotinylated monoclonal antibody. The complex BSA-hapten-antibody-biotin was thereafter probed with STP40.

The results which are shown in FIG. 15 reveal that even at concentrations as low as a few tens of picomolars of antibody, detection was successful.

In order to obtain another approximation of the detection abilities of the method of the present invention, equilibrium equations were used to calculate how many hapten-antibody complexes are expected to form under the experimental conditions employed. The affinity constant (K=4 nM) was assumed to be the same as when both hapten and antibody are free in solution, based on 2 independent measurements conducted in Fluorescence and ELISA assays (see methods). The efficiency of attachment of the BSA-hapten to the substrate, taking into account the binding capacity of the aldehyde groups was assumed to be <30%. Then the ratio of the number of complexes detected by the gold colloids to the maximal number of complexes expected to exist on the glass was calculated. A ratio close to 1:10 was obtained, indicating that method detects about one of every ten complexes that are actually formed on the slide. In view of the calculations above, it is again anticipated that using smaller gold colloids this ratio will substantially improve.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-105. (canceled)

106. A method for detecting binding between all least one first member of a binding pair and at least one corresponding second member of the binding pair, the method comprising:
interacting a solid support auto which said at least one first member of the binding pair is immobilized and arrayed with said at least one corresponding second member of the binding pair,
wherein said at least one corresponding second member of the binding pair is at least one of:
(a) directly tagged; and
(b) indirectly tagged;
with a heavy atom; and
determining a spatial distribution of said heavy atom over a surface of the solid support so as to detect binding between said al least one first member of the binding pair and said at least one corresponding second member of the binding pair.

107. The method of claim 106, wherein said determining the spatial distribution of said heavy atom over said surface of the solid support has a dynamic range of linearity of at least four orders-of-magnitude.

108. The method of claim 106, wherein said determining the spatial distribution of said heavy atom over said surface of the solid support is at a sensitivity of detection equal to at least 1 in 10 binding events.

109. The method of claim 108, wherein said sensitivity is equal to at least 1 in 5 binding events.

110. The method of claim 109, wherein said sensitivity is about 1 in 1 binding events.

111. The method of claim 106, wherein said determining the spatial distribution of said heavy atom over said surface of the solid support is at a signal-to-noise ratio of more than 20.

112. The method of claim 106, wherein said determining the spatial distribution of said heavy atom over said surface of the solid support is at a signal-to-noise ratio of more than 50.

113. The method of claim 106, wherein said determining the spatial distribution of said heavy atom over said surface of the solid support is at a signal-to-noise ratio of more than 80.

114. The method of claim 106, wherein said binding pair is selected from the group consisting of antigen-antibody, antibody-antigen, hapten-antibody, antibody-hapten, nucleic acid-complementary nucleic acid, nucleic acid-substantially complementary nucleic acid, ligand-receptor, receptor-ligand, enzyme-substrate, substrate-enzyme, enzyme-inhibitor and inhibitor-enzyme.

115. The method of claim 106, wherein said determining the spatial distribution for said heavy atom over said surface of the solid support is by particle scattering.

116. The method of claim 106, wherein said determining the spatial distribution for said heavy atom over said surface of the solid support is by electron scattering.

117. The method of claim 106, wherein said heavy atom is selected from the group consisting of gold, silver and iron.

118. A method for detecting binding between at least one first member of a binding pair and at least one corresponding second member of the binding pair, the method comprising:
interacting a solid support onto which said at least one first member of the binding pair is immobilized and arrayed with said at least one corresponding second member of the binding pair; and
determining a spatial distribution of said at least one corresponding second member of the binding hair at a signal-to-noise ratio of more than 10.

119. The method of claim 118, wherein said at least one first member comprises at least one biological molecule is selected from the group consisting of a protein, a glycoprotein, a nucleic acid and a carbohydrate.

120. The method of claim 118, wherein said at least one second member comprises at least one macromolecule of a known identity selected from the group consisting of proteins, glycoproteins, nucleic acids and carbohydrates.

121. A method for at least one of identifying and quantifying at least one biological molecule in a sample, the method comprising;
contacting the sample with a microarray presenting an addressable array of macromolecules of known identities under conditions so as to allow binding between said at least one biological molecule and said macromolecules of known identities; and
detecting a spatial distribution of said at least one biological molecule over a surface of said microarray at a sensitivity equal to at least 1 in 10 binding events so as to provide at least one of an identification and a quantification of the least one biological molecule in the sample.

122. The method of claim 121, wherein said at least one biological molecule is selected from the group consisting of a protein, a glycoprotein, a nucleic acid and a carbohydrate.

123. The method of claim 121, wherein said macromolecules of known identities are selected from the group consisting of proteins, glycoproteins, nucleic acids and carbohydrates.

124. The method of claim 121, wherein said detecting the spatial distribution of said at least one biological molecule over said surface of said microarray has a dynamic range of linearity of at least four orders-of-magnitude.

125. The method of claim 121, wherein said detecting the spatial distribution of said at least one biological molecule over said surface of said microarray has a signal-to-noise ratio of more than 20.

126. The method of claim 121, wherein said detecting the spatial distribution of said at least one biological molecule over said surface of said microarray is by directly or indirectly tagging said at least one biological molecule with at least one heavy atom and obtaining a particle scattering image of a spatial distribution of said at least one heavy atom.

127. The method of claim 121, wherein the signal-to-noise ratio is greater thin 20.

128. A method for at least one of identifying and quantifying at least one biological molecule in a sample, the method comprising:
attaching biological molecules present in the sample to a solid support;
contacting said solid support with at least one macromolecule of a known identity under conditions so as to allow binding between said at least one biological molecule and said at least one macromolecule of known identity; and
detecting a level of binding between said at least one biological molecule and said at least one macromolecule of known identity by directly or indirectly tagging said at least one macromolecule of known identity with at least one heavy atom and obtaining a particle scattering image of a spatial distribution of said at least one heavy atom, thereby providing at least one of an identification and a quantification of the least one biological molecule in the sample.

129. The method of claim 128, wherein said at least one biological molecule is selected from the group consisting of a protein, a glycoprotein, a nucleic acid and a carbohydrate.

130. The method of claim 128, wherein said at least one macromolecule of known identity is selected from the group consisting of a protein, a glycoprotein, a nucleic acid and a carbohydrate.

131. The method of claim 128, wherein said detecting the level of binding between said at least one biological molecule and said at least one macromolecule of known identity has a dynamic range of linearity of at least four orders-of-magnitude.

132. The method of claim 128, wherein said detecting the level of binding between said at least one biological molecule and said at least one macromolecule of known identity is at a sensitivity equal to at least 1 in 10 binding events.

133. The method of claim 128, wherein said detecting the level of binding between said at least one biological molecule and said at least one macromolecule of known identity is at a signal-to-noise ratio of more than 20.

134. A method for at least one of identifying and quantifying biological molecules in a preparation, the method comprising:
localizing and tagging the biological molecules in the preparation;
preparing the preparation for vacuum;
loading the preparation into the specimen chamber of an electron beam device;
irradiating the preparation with an electron beam, thus obtaining an image of the tags; and
analyzing the image of the biological molecules by image analysis software so as to provide at least one of an identification and a quantification of the biological molecules in said preparation.

135. A method for at least one of identifying and quantifying biological molecule in a preparation, the method comprising:
localizing and tagging the biological molecules in the preparation;
lading the preparation into the specimen clamber of an electron beam device;
irradiating the preparation with an electron beam, thus obtaining an image of the tags; and
analyzing the image of the biological molecules by image analysis software so as to provide at least one of an identification and a quantification of the biological molecules in sail preparation.

136. An apparatus for analysis of at least two samples comprising biological molecules, comprising:
an electron source to provide an electron beam;
a charged particle beam column to deliver and scan an electron beam from said electron source on the surface of a first sample of said at least two samples;
a vacuum system including a first and a second chamber in each of which pressurization can be performed independently to permit loading or unloading of a second sample of said at least two samples in one chamber while simultaneously inspecting said first sample;
at least one electron detector;
a measuring system for measuring X-ray spectrum;
a continuously moving x-y stage disposed to receive said second sample and to provide at least one degree of motion to said second sample while the first sample is being scanned; and
an image analysis system for carrying, out image analysis of the molecules of the first and second samples.
